(19) United States
(12) Patent Application Publication
(10) Pub. No.: US 2024/0382097 A1
(43) Pub. Date: Nov. 21, 2024

(54) APPARATUS AND METHOD FOR MEASURING PHYSIOLOGICAL PARAMETERS OF MAMMAL SUBJECT USING EASILY REMOVABLE FLEXIBLE ELECTRONICS AND APPLICATIONS THEREOF

(71) Applicant: NORTHWESTERN UNIVERSITY, Evanston, IL (US)

(72) Inventors: Sung Soo Kwak, Evanston, IL (US); Shuai Xu, Bala Cynwyd, PA (US); Ha UK Chung, Evanston, IL (US); Hyoyoung Jeong, Evanston, IL (US); John A. Rogers, Wilmette, IL (US)

(21) Appl. No.: 18/691,924

(22) PCT Filed: Sep. 15, 2022
(86) PCT No.: PCT/US2022/043575

§ 371 Date: Mar. 14, 2024

Related U.S. Application Data
(60) Provisional application No. 63/244,277, filed on Sep. 15, 2021.

(51) Int. Cl. A61B 5/0205; A61B 5/00; A61B 5/01; A61B 5/02; A61B 5/021; A61B 5/024; A61B 5/08; A61B 5/113; A61B 5/145; A61B 5/257; A61B 5/263; A61B 5/28; A61B 5/296

(52) U.S. Cl. CPC A61B 5/02055; A61B 5/257; A61B 5/263; A61B 5/28; A61B 5/296; A61B 5/6833; A61B 5/7225; A61B 5/742; A61B 5/01; A61B 5/02007; A61B 5/021; A61B 5/02405; A61B 5/0816; A61B 5/0823; A61B 5/113; A61B 5/14542; A61B 5/4812; A61B 5/4815; A61B 5/4818; A61B 2560/0214; A61B 2560/045; A61B 2560/0468; A61B 2562/0204; A61B 2562/0219; A61B 2562/0271; A61B 2562/164; A61B 2562/166; A61B 2562/227

(57) ABSTRACT

This invention relates to apparatuses and methods for non-invasively measuring physiological parameters of a mammal subject using easily removable flexible electronics, and applications of the same. Specifically, a novel sensor class with a pre-curved architecture and strategically located perforations that may be used in apparatuses and methods for measuring physiological parameters of a mammal subject. Further, adhesive layers are used to attach the sensor systems on the skin of the mammal subject, and each adhesive layer is switchable chemically or physically between an adhesive state and a non-adhesive state, allowing easy removal of the corresponding sensor system from the skin of the mammal subject when being switched to the non-adhesive state.

### CROSS-REFERENCE TO RELATED APPLICATIONS

This PCT application claims priority to and the benefit of U.S. Provisional Patent Application Ser. No. 63/244,277, which was filed Sep. 15, 2021. The content of the application is incorporated herein by reference in its entirety.

This PCT application is related to a co-pending PCT patent application entitled “APPARATUS AND METHOD FOR MEASURING PHYSIOLOGICAL PARAMETERS OF MAMMAL SUBJECT AND APPLICATIONS OF SAME”, by John A. Rogers et al., with International Application No. PCT/US19/59131, a co-pending PCT patent application entitled “SENSOR NETWORK FOR MEASURING PHYSIOLOGICAL PARAMETERS OF MAMMAL SUBJECT AND APPLICATIONS OF SAME”, by John A. Rogers et al., with International Application No. PCT/US19/59156, a co-pending U.S. patent application entitled “APPARATUS AND METHOD FOR NON-INVASIVELY MEASURING BLOOD PRESSURE OF MAMMAL SUBJECT”, by John A. Rogers et al., with U.S. Application No. 16/670, 161, and a co-pending PCT patent application entitled “APPARATUS AND METHOD FOR NON-INVASIVELY MEASURING PHYSIOLOGICAL PARAMETERS OF MAMMAL SUBJECT AND APPLICATIONS THEREOF”, by John A. Rogers et al., with International Application No. PCT/US19/59190, each of which is filed on Oct. 31, 2019, and is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### FIELD OF THE INVENTION

The present invention relates generally to healthcare and vital sign monitoring, and more particularly to apparatuses and methods for non-invasively measuring physiological parameters of a mammal subject using easily removable flexible electronics, and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Soft, flexible vital signs monitoring systems have shown significant promise for continuous vital signs monitoring. However, ultra-fragile skin and patient comfort dictates additional needs. First, there remains a continuous need to increase mechanical compliance to reduce skin contact stress for these systems. Second, there remains a need to visually interrogate the skin health beneath these sensors. Third, peel force remains a significant problem in the context of fragile skin. The removal of sensors and their corresponding adhesives can lead to epidermal skin stripping.

Therefore, a heretofore unaddressed need exists in the art to address the aforementioned deficiencies and inadequacies.

### SUMMARY OF THE INVENTION

Certain aspects of the invention relate to apparatuses and methods for measuring physiological parameters of mammal subjects using easily removable flexible electronics, and applications of the same.

In one aspect, the invention relates to an apparatus for measuring physiological parameters of a mammal subject. In certain embodiments, the apparatus includes: a plurality of sensors configured to be time-synchronized and in communication with each other wirelessly and bidirectionally, wherein each of the sensors is bendable such that each of the sensors is configured to have a pre-curved architecture with a non-zero curvature, and each of the sensors is configured to detect a vital sign of the mammal subject and generate a corresponding one of the physiological parameters; and a plurality of adhesive layers, configured to be disposed between the sensors and a skin of the mammal subject correspondingly, wherein each of the adhesive layers is switchable chemically or physically between an adhesive state and a non-adhesive state, such that, for a corresponding sensor of the sensors and a corresponding adhesive layer of the adhesive layers, the corresponding adhesive layer is configured to attach the corresponding sensor to a corresponding location on the mammal subject in the adhesive state, and to allow removal of the corresponding sensor system from the skin of the mammal subject when being switched to the non-adhesive state; wherein the non-zero curvature of the corresponding sensor is configured to be adjustable based on a shape of the corresponding location on the mammal subject.

In another aspect, the invention is related to a method of measuring physiological parameters of a mammal subject, which includes: attaching, by a plurality of adhesive layers in an adhesive state, a plurality of sensors on the mammal subject, wherein the adhesive layers are correspondingly disposed between the sensor systems and a skin of the mammal subject, each of the adhesive layers is switchable chemically or physically between the adhesive state and a non-adhesive state, the sensors are configured to be time-synchronized and in communication with each other wirelessly and bidirectionally, each of the sensors is bendable such that each of the sensors is configured to have a pre-curved architecture with a non-zero curvature, and each of the sensors is configured to monitor one of the physiological parameters; measuring, by the sensors, the physiological parameters of the mammal subject; and in response to a need to remove a corresponding sensor of the sensors from a corresponding location on the mammal subject, switching chemically or physically a corresponding adhesive layer of the adhesive layers to the non-adhesive state to remove the corresponding sensor from the skin of the mammal subject, wherein the non-zero curvature of the corresponding sensor is adjustable based on a shape of the corresponding location on the mammal subject.

In certain embodiments, each of the sensors is at least partially formed by a bendable shape-memory alloy (SMA).

In certain embodiments, each of the sensors is formed by at least three island regions and at least two flexible and stretchable interconnects, each of the at least two flexible and stretchable interconnects is interconnected between two adjacent island regions of the at least three island regions, and each of the at least two flexible and stretchable interconnects is formed by the SMA.

In certain embodiments, each of the sensors is an epidermal electronic system (EES) comprising: a middle circuit board layer including a plurality of electronic components disposed on each of the at least three island regions, wherein the at least two flexible and stretchable interconnects are electrically connected to different ones of the electronic components; and a top elastomeric encapsulation layer and a bottom elastomeric encapsulation layer sandwiching the middle circuit board layer, wherein the bottom elastomeric encapsulation layer forms a tissue-facing surface attached to the mammal subject, and the top elastomeric encapsulation layer forms an environment-facing surface.

In certain embodiments, the SMA is a nickel-based SMA, an iron-based SMA, a copper-based SMA, or a combination thereof. In one embodiment, the SMA is nitonol.

In certain embodiments, each of the sensors includes a plurality of perforations, such that the perforations of the corresponding sensor are configured to enable direct access to the corresponding adhesive layer.

In certain embodiments, for each of the sensors, the perforations are located to allow direct visualization of the skin of the mammal subject underlying the sensors without removal of the sensors.

In certain embodiments, each of the adhesive layers is switchable chemically from the adhesive state to the non-adhesive state by applying a liquid or a chemical solution directly to the adhesive layers through the perforations.

In certain embodiments, the liquid or the chemical solution includes: water, normal saline, a solution with a certain level of pH value, a solution for dissolving the adhesive layers through thiol-disulfide exchange reactions, retro-Michael reactions, or retro Diels-Alder reactions, and a glucose solution for dissolving the adhesive layers.

In certain embodiments, each of the adhesive layers is switchable physically from the adhesive state to the non-adhesive state through a thermal process or light.

In certain embodiments, each of the adhesive layers is formed by a hydrogel adhesive, hydrocolloid adhesives, a polymeric film, fiber adhesives, or an acrylic adhesive.

In certain embodiments, each of the sensors is configured to detect the vital sign as a signal including one of: an electrical signal related to at least one of electrocardiography (ECG) and electromyography (EMG) technology; a mechanical signal related to movement, respiration and arterial tonometry; an acoustic signal related to vocal cord vocalization, respiratory sound and heart sound; and an optical signal related to at least one of blood oxygenation and blood pressure.

In certain embodiments, each of the sensors further comprises a power supply, and the power supply is an embedded power supply or a detachable modular power supply.

In certain embodiments, the apparatus further includes a microcontroller unit (MCU) configured to be in wireless communication with the plurality of sensors, and configured to receive the physiological parameters of the mammal subject from the sensors and to display the physiological parameters of the mammal subject.

In certain embodiments, each of the sensors is configured to be in wireless communication with the MCU via a near field communication (NFC) protocol, or Bluetooth protocol.

In certain embodiments, each of the sensors comprises a magnetic coil in compliance with the NFC protocol to allow wireless data transmission and wireless power transmission through a single link.

In certain embodiments, each of the plurality of sensors further comprises one or more of: an accelerometer for position or movement monitoring; and a temperature sensor for measuring temperature.

In certain embodiments, the physiological parameters of the mammal subject comprise one or more of: heart rate, heart rate variability, heart sounds, blood pressure, chest wall displacement, electromyography, electrocardiogramanation, respiratory rate, respiratory effort, respiratory cadence, tidal volume, coughing, snoring, sneezing, throat clearing, wheezing, apnea, hypoapnea, physical activity, core body position, peripheral limb position, scratching, vocalizations, rubbing, walking, sleep quality, sleep time, wake time upon sleeping, skin temperature, core body temperature, and a combination thereof.

In one embodiment, the switching of the corresponding adhesive layer the non-adhesive state is performed by: applying a thermal process or light to the corresponding adhesive layer.

In certain embodiments, the mammal subject is a human subject or a non-human subject.

In certain embodiments, the sensors are configured to be in wireless communication with a microcontroller unit (MCU). In one embodiment, the method further includes: receiving, at the MCU, the physiological parameters of the mammal subject; and displaying, at the MCU, the physiological parameters of the mammal subject.

Yet another aspect of the invention relates to an apparatus for measuring physiological parameters of a mammal subject. In certain embodiments, the apparatus includes: a plurality of sensor systems configured to be time-synchronized and in communication with each other wirelessly and bidirectionally, wherein each of the sensor systems comprises a flexible printed circuit board (fPCB) with a pre-curved perforated architecture and at least one sensor disposed on the fPCB, and the at least one sensor is configured to detect a vital sign of the mammal subject and generate a corresponding one of the physiological parameters; and a plurality of adhesive layers, configured to be disposed between the sensor systems and a skin of the mammal subject correspondingly, wherein each of the adhesive layers has a perforated architecture and is switchable chemically or physically between an adhesive state and a non-adhesive state, such that, for a corresponding sensor system of the sensor systems and a corresponding adhesive layer of the adhesive layers, the corresponding adhesive layer is configured to attach the corresponding sensor system to the mammal subject in the adhesive state, and to allow removal of the corresponding sensor system from the skin of the mammal subject when being switched to the non-adhesive state. Yet a further aspect of the invention relates to a method of measuring physiological parameters of a mammal subject, which includes: attaching, by a plurality of adhesive layers in an adhesive state, a plurality of sensor systems on the mammal subject, wherein the adhesive layers are correspondingly disposed between the sensor systems and a skin of the mammal subject, each of the adhesive layers has a pre-curved perforated architecture and is switchable chemically or physically between the adhesive state and a non-adhesive state, the sensor systems are configured to be time-synchronized and in communication with each other wirelessly and bidirectionally, and each of the sensor systems comprises a flexible printed circuit board (fPCB) with a perforated architecture and at least one sensor disposed on the fPCB to monitor one of the physiological parameters; measuring, by the sensor systems, the physiological parameters of the mammal subject; and in response to a need to remove a corresponding sensor system of the sensor systems, switching chemically or physically a corresponding adhesive layer of the adhesive layers to the non-adhesive state to remove the corresponding sensor system from the skin of the mammal subject.

In certain embodiments, each of the sensor systems includes a plurality of perforations, such that the perforations of the corresponding sensor system are configured to enable direct access to the corresponding adhesive layer.

In certain embodiments, for each of the sensor systems, the perforations are located to allow direct visualization of the skin of the mammal subject underlying the sensor systems without removal of the sensor systems.

In certain embodiments, each of the sensor systems is an epidermal electronic system (EES) comprising: a middle circuit board layer including a plurality of electronic components, and a plurality of flexible and stretchable interconnects electrically connected to different electronic components; and a top elastomeric encapsulation layer and a bottom elastomeric encapsulation layer sandwiching the middle circuit board layer, wherein the bottom elastomeric encapsulation layer forms a tissue-facing surface attached to the mammal subject, and the top elastomeric encapsulation layer forms an environment-facing surface.

In certain embodiments, the middle circuit board layer is formed by a foldable electronic board, and the plurality of electronic components and the plurality of flexible and stretchable interconnects are disposed on the foldable electronic board.

In certain embodiments, the perforations of each of the sensor systems are formed by corresponding perforations formed on each of the top elastomeric encapsulation layer, the middle circuit board layer and the elastomeric encapsulation layer, the corresponding perforations formed on the middle circuit board layer exists between the electronic components, and the corresponding perforations formed on the top elastomeric encapsulation layer and the bottom elastomeric encapsulation layer integrate and align with the corresponding perforations formed on the middle circuit board layer.

In certain embodiments, each of the adhesive layers is switchable chemically from the adhesive state to the non-adhesive state by applying a liquid or a chemical solution directly to the adhesive layers through the perforations.

In certain embodiments, the liquid or the chemical solution includes: water, normal saline, a solution with a certain level of pH value, a solution for dissolving the adhesive layers through thiol-disulfide exchange reactions, retro-Michael reactions, or retro Diels-Alder reactions, and a glucose solution for dissolving the adhesive layers.

In certain embodiments, each of the adhesive layers is switchable physically from the adhesive state to the non-adhesive state through a thermal process or light.

In certain embodiments, each of the adhesive layers is formed by a hydrogel adhesive, hydrocolloid adhesives, a polymeric film, fiber adhesives, or an acrylic adhesive.

In certain embodiments, the sensor is configured to detect the vital sign as a signal including one of: an electrical signal related to at least one of electrocardiography (ECG) mand electromyography (EMG) technology; a mechanical signal related to movement, respiration and arterial tonometry; an acoustic signal related to vocal cord vocalization, respiratory sound and heart sound; and an optical signal related to at least one of blood oxygenation and blood pressure.

In certain embodiments, each of the sensor systems further comprises a power supply, and the power supply is an embedded power supply or a detachable modular power supply.

In certain embodiments, the apparatus further includes a microcontroller unit (MCU) configured to be in wireless communication with the plurality of sensor systems, and configured to receive, from the sensor systems, and to display the physiological parameters of the mammal subject. In one embodiment, each of the sensor systems is configured to be in wireless communication with the MCU via a near field communication (NFC) protocol, or Bluetooth protocol. In certain embodiments, each of the sensor systems comprises a magnetic coil in compliance with the NFC protocol to allow wireless data transmission and wireless power transmission through a single link.

In certain embodiments, each of the plurality of sensor systems further comprises one or more of: an accelerometer for position or movement monitoring; and a temperature sensor for measuring temperature.

In certain embodiments, the physiological parameters of the mammal subject comprise one or more of: heart rate, heart rate variability, heart sounds, blood pressure, chest wall displacement, electromyography, electrocardiogramanation, respiratory rate, respiratory effort, respiratory cadence, tidal volume, coughing, snoring, sneezing, throat clearing, wheezing, apnea, hypoapnea, physical activity, core body position, peripheral limb position, scratching, vocalizations, rubbing, walking, sleep quality, sleep time, wake time upon sleeping, skin temperature, core body temperature, and a combination thereof.

In certain embodiments, the mammal subject is a human subject or a non-human subject.

In certain embodiments, the switching of the corresponding adhesive layer the non-adhesive state is performed by: applying a liquid or a chemical solution directly to the corresponding adhesive layer through the perforations of the corresponding sensor system.

In certain embodiments, the switching of the corresponding adhesive layer the non-adhesive state is performed by: applying a thermal process or light to the corresponding adhesive layer.

In certain embodiments, the sensor systems are configured to be in wireless communication with a microcontroller unit (MCU). In one embodiment, the method further includes: receiving, at the MCU, the physiological parameters of the mammal subject; and displaying, at the MCU, the physiological parameters of the mammal subject.

In yet a further aspect, the invention relates to a non-transitory tangible computer-readable medium storing instructions which, when executed by one or more processors, cause the method as disclosed above to be performed.

Still another aspect of the invention relates to an apparatus for measuring physiological parameters of a mammal subject, which includes: at least two sensors configured to be in communication with each other wirelessly and bidirectionally in operation, wherein each of the two sensors is bendable such that each of the sensors is configured to have a pre-curved architecture with a non-zero curvature.

In certain embodiments, each of the two sensors is at least partially formed by a bendable shape-memory alloy (SMA).

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein. The drawings described below are for illustration purposes only. The drawings are not intended to limit the scope of the present teachings in any way.

FIG. 1 schematically shows a functional block diagram of an apparatus according to certain embodiments of the present invention.

FIG. 2 schematically shows a sensor system being attached to the skin of a mammal subject by the adhesive layer according to certain embodiments of the present invention.

FIG. 3A schematically shows (i) an explosive view of a sensor system, (ii) a middle circuit layer of the sensor system, and (iii) an explosive view of the layers of the sensor system according to certain embodiments of the present invention.

FIG. 3B schematically shows an exemplary middle circuit layer of the sensor system according to certain embodiments of the present invention.

FIG. 3C schematically shows an exemplary sensor system with perforations having specific configurations, sizes and locations according to certain embodiments of the present invention.

FIG. 3D schematically shows different exemplary sensor systems (A) and (B) with perforations having different configurations, sizes and locations according to certain embodiments of the present invention.

FIG. 3E schematically shows deformation of the layers of the sensor system according to certain embodiments of the present invention.

FIG. 4A schematically shows switching an adhesive layer chemically from the adhesive state to the non-adhesive state according to certain embodiments of the present invention.

FIG. 4B schematically shows switching an adhesive layer physically from the adhesive state to the non-adhesive state according to certain embodiments of the present invention.

FIG. 5 shows a flowchart of a method of measuring physiological parameters of a mammal subject according to certain embodiments of the present invention.

FIG. 6A shows an exploded schematic view of a soft, perforated wireless device with a rechargeable battery for measuring electrocardiograms (ECGs) and skin temperature, and for capturing tri-axis accelerometry data according to certain embodiments of the present invention.

FIG. 6B shows (a) a block diagram of the operational scheme of the device as shown in FIG. 6A, and (b) images of the device as shown in FIG. 6A on the chest of a realistic model of a neonate according to certain embodiments of the present invention.

FIG. 7 schematically shows the design and construction of a perforated device platform according to certain embodiments of the present invention.

FIG. 8 schematically shows the serpentine interconnects used in a perforated device according to certain embodiments of the present invention.

FIG. 9 schematically shows the results of modeling of the mechanical properties of serpentine interconnects for pre-compression according to certain embodiments of the present invention.

FIG. 10 schematically shows the computed distributions of strain in the copper layers of the serpentine interconnects associated with pre-compression according to certain embodiments of the present invention.

FIG. 11 schematically shows the modeling of the strain distributions in the copper layers of the serpentine interconnects for stretching deformations according to certain embodiments of the present invention.

FIG. 12 schematically shows a summary of the process for encapsulation of a perforated device according to certain embodiments of the present invention.

FIG. 13A schematically shows the mechanical characterization results and images of a soft, perforated, wireless vital signs monitoring device under various mechanical deformations according to certain embodiments of the present invention, where (A) shows images of a representative device during (i) parallel bending, (ii) horizontal bending, (iii) twisting and (iv) stretching, and (B) shows simulation results for the deformed geometries and strain distributions in the copper layer of the electronic system.

FIG. 13B schematically shows the mechanical characterization results and images of the monitoring device as shown in FIG. 13A, where (A) shows simulation results for the deformed geometries and strain distributions in entire encapsulated device during corresponding deformations, and (B) shows comparisons of moment-angle and force-strain responses for holey and non-holey device designs according to certain embodiments of the present invention.

FIG. 14 schematically shows the computed strain distribution in the copper layers of the serpentine interconnect for bending deformations in the parallel direction according to certain embodiments of the present invention.

FIG. 15 schematically shows the computed strain distribution in the copper layers of the serpentine interconnect for bending deformations in the horizontal direction according to certain embodiments of the present invention.

FIG. 16 schematically shows the computed strain distributions in the copper layers of the serpentine interconnect for twisting deformations according to certain embodiments of the present invention.

FIG. 17 schematically shows comparisons of computed strain distributions in the encapsulated device with and without holes under various deformations according to certain embodiments of the present invention.

FIG. 18 shows a table of comparison of computed strain distributions and strain reductions in the encapsulated devices with perforated and non-perforated designs for various deformations according to certain embodiments of the present invention.

FIG. 19 schematically shows the comparisons of computed moment-angle and force-strain responses for perforated and non-perforated device designs for deformations such as parallel bending, horizontal bending, twisting and stretching according to certain embodiments of the present invention.

FIG. 20 schematically shows the computed results for shear and normal stress at the interface between perforated and non-perforated device designs and skin according to certain embodiments of the present invention.

FIG. 21 schematically shows the pre-curved perforated ECG devices with different curvatures according to certain embodiments of the present invention.

FIG. 22 schematically shows a summary of the process for pre-curved encapsulation of a perforated device according to certain embodiments of the present invention.

FIG. 23 schematically shows computed shear and normal stress at the interface between skin and perforated devices after 15% tangential stretch according to certain embodiments of the present invention.

FIG. 24 schematically shows water triggered soft release of the hydrogel adhesive according to certain embodiments of the present invention.

FIG. 25 shows a photograph of the adhesion test setup for the hydrogel adhesive, using rectangular test structure (10×30 mm²) attached on a slide glass according to certain embodiments of the present invention.

FIG. 26 shows the comparison of diffusion coefficients of a hydrogel adhesive (KM 40A) for water at 25° C. and 35° C. according to certain embodiments of the present invention.

FIG. 27 shows the computed diffusion time as a function distance between the holes according to certain embodiments of the present invention.

FIG. 28 shows the modeling results of diffusion for different hydrogel adhesive designs at 25° C. and 35° C. according to certain embodiments of the present invention.

FIG. 29 schematically shows a photograph of a hydrogel adhesive design with holes and microchannels according to certain embodiments of the present invention.

FIG. 30 shows water introduced into the holes and water passing through the microchannels in a hydrogel adhesive for a perforated device with microchannel design according to certain embodiments of the present invention.

FIG. 31 shows the computed swelling model of water diffusion into the hydrogel adhesive as a function of time, water temperature, and adhesive design according to certain embodiments of the present invention, where (i) shows the perforated design at 25° C., (ii) shows the perforated design at 35° C. and (iii) shows the perforated design with microchannels at 35° C.

FIG. 32 shows a table of the time to swell completely depending on hydrogel designs and water temperature according to certain embodiments of the present invention.

FIG. 33 shows the peeling test for a hydrogel adhesive on skin according to certain embodiments of the present invention.

FIG. 34 schematically shows the measurements of breathability and demonstrations of visual inspection through the openings in a perforated device according to certain embodiments of the present invention.

FIG. 35 schematically shows the testing protocol for evaluating water evaporation from the hydrogel adhesive according to certain embodiments of the present invention.

FIG. 36 shows representative data collected from a perforated device according to certain embodiments of the present invention.

FIG. 37A shows representative photographs of a perforated device with the newborns and a neonatal patient in an operating NICU according to certain embodiments of the present invention.

FIG. 37B shows clinical demonstrations of the perforated device with the newborns and the neonatal patient as shown in FIG. 37A, where (A) shows representative ECG, and SCG waveforms recorded from the patient using the device; (B) shows comparison of HR determined with the device and with a gold standard system; and (C) shows corresponding Bland-Altman plot for HR according to certain embodiments of the present invention.

FIG. 38 schematically shows a sensor with a pre-curved architecture according to certain embodiments of the invention.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this specification will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term are the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures. is turned over, elements described as being on the “lower” side of other elements would then be oriented on “upper” sides of the other elements. The exemplary term “lower”, can, therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this specification, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and this specification, and will not be interpreted in an idealized or overly formal sense unless expressly so defined herein.

As used in this specification, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this specification, the phrase “at least one of A, B, and C” should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in a different order (or concurrently) without altering the principles of the invention.

As discussed above, soft, flexible vital signs monitoring systems have shown significant promise for continuous vital signs monitoring, but ultra-fragile skin and patient comfort dictates additional needs. To address these additional needs, methods and technologies that can reduce peel force to near zero or zero would offer significant clinical utility. To date, vital signs monitoring systems obscure the underlying skin, lack mechanical compliance, and unable to offer methods to reduce peel force for ultra-fragile skin. In certain aspects of this invention, the inventors propose a novel sensor class with strategically located perforations that may be used in apparatuses and methods for measuring physiological parameters of a mammal subject. The novel sensor class offers the following advantages: greater mechanical compliance with specific articulations that follow physiological movement, greater mechanical compliance with specific articulations that enable reduced peel force (e.g. enables peeling directionality that reduces skin contact stress), more controlled deformations enabling greater overall mechanical robustness, ability to directly observe underlying skin, perforations that allow for avoidance of rashes or wounds, and perforations that enable facilitate physical or chemical means that eliminate or reduce adhesive strength of underlying adhesive to reduce peel force. In addition, these perforations may also significant reduce sensor weight-which would hold clinical relevance in monitoring ultra-low birth weight neonates. Applications of the apparatuses and/or methods may include, without being limited thereto, remote patient monitoring, critical care monitoring (specifically vulnerable populations such as the NICU), and assisted living monitoring (specifically vulnerable populations such as the elderly).

Continuous monitoring of vital signs is an essential aspect of operations in neonatal and pediatric intensive care units (NICUs and PICUs), of particular importance to extremely premature and/or critically ill patients. Each year, more than 480,000 infants and children in the United States are admitted to intensive care units (ICUs). Those under one year of age, particularly very low-birth-weight premature infants, suffer from high rates of morbidity and mortality. For these fragile patients, real-time monitoring of their vital signs represents an essential aspect of care. Traditional systems for such purposes in the neonatal and pediatric ICUs (NICUs and PICUs) involve multiple electrodes and sensors that attach to various parts of the body using adhesive tapes. Hard wires form interconnections to external electronic processing and storage units. These platforms can provide high quality data but they have significant disadvantages. For neonates and pediatric patients with immature skin, iatrogenic injuries and subsequent scarring can result from the electrodes/sensors and adhesives. This hardware also frustrates natural movements, it creates practical difficulties in feeding, changing diapers and bathing, and it limits opportunities for physical bonding with parents through skin-to-skin contact (i.e., kangaroo care, KC).

Soft, wireless electronic devices can overcome these and other drawbacks of traditional monitoring equipment, to enable safe and effective care for vulnerable patients. The inventors recently reported two such types of technologies and demonstrated their use in NICU and PICU facilities. The most advanced systems operate in a battery-free manner, through devices that have ultrathin, stretchable, ‘skin-like’ characteristics. A time-synchronized pair can capture heart rate (HR), heart rate variability (HRV), respiration rate (RR), blood oxygen saturation (SpO2), pulse wave velocity and skin temperature at the chest and at a limb, all with clinical-grade levels of accuracy. The thin, low modulus properties and wireless operation of these devices allow robust bonding to the skin with interfacial adhesives that require bonding strengths that are more than ten times less than those needed with conventional wired sensors, thereby reducing the probability for iatrogenic skin injuries during removal. Additional important features include sparse architectures that provide optical transparency, for visual inspection of the skin interface, and electromagnetic designs that allow use of magnetic resonance imaging and X-ray computed tomography, for various diagnostic purposes. A limitation of this technology is that the mechanical fragility of the devices leads to practical difficulties in multiple cycles of application, removal and sterilization, particularly relevant for use in the home or in lower and middle income countries. Also, the near-field communication (NFC) schemes for wireless power delivery and communication require reliable wall-plug power, RF transmission hardware and specialized electronics. Furthermore, the protocols can only support operating ranges and data transfer rates of up to 30 cm and a few hundred Hz, respectively. Related constraints prevent monitoring modalities that extend beyond the current clinical standard of care.

A second study introduced an alternative, complementary platform based on Bluetooth technology and small, rechargeable batteries, with capabilities that bypass these and other limitations of the NFC system. Advanced functional modes support capabilities in monitoring movements and changes in body orientation, in capturing seismocardiograms (SCGs) and in recording vocal biomarkers and other sounds of body functions (e.g. respiration, gastrointestinal processes, etc), each of relevance for early detection of complications related to birth trauma, brain injury and/or or pain stress. Relative to the NFC platforms, these systems have larger thicknesses, masses and effective mechanical properties, they require stronger adhesives to maintain robust bonding to the skin, particularly at highly curved regions on the most premature babies, and they have an opaque construction that prevents optical inspection of the skin/device interface.

To address the disadvantages, certain embodiments of the invention address the issues through the use of optimized materials in open, or ‘holey’ (i.e., perforated), device architectures and with pre-curved layouts. The holes, with strategic locations and dimensions defined by finite element analysis (FEA), enhance the flexibility and stretchability of the devices in various directions that are important to practical use. Perhaps more significantly, these features also provide multiple points of access for introducing warm water to the skin interface. The consequent swelling of hydrogel adhesives molded with microfluidic channel structures dramatically reduces the bonding strength, in some cases to negligible values, to facilitate device removal. Additional aspects of this design include (1) visual access to the underlying skin, for clinical inspection for irritation or allergic reactions, and (2) mechanisms for enhanced evaporation of water released through natural transepidermal mechanisms or sweating. Precurved shapes tailored to the match the surfaces of desired mounting locations minimize bending forces and corresponding stresses at the skin interface. Systematic studies of devices designed to monitor a full range of conventional and unconventional metrics of physiological health reveal all of the key aspects of these advanced materials and design ideas.

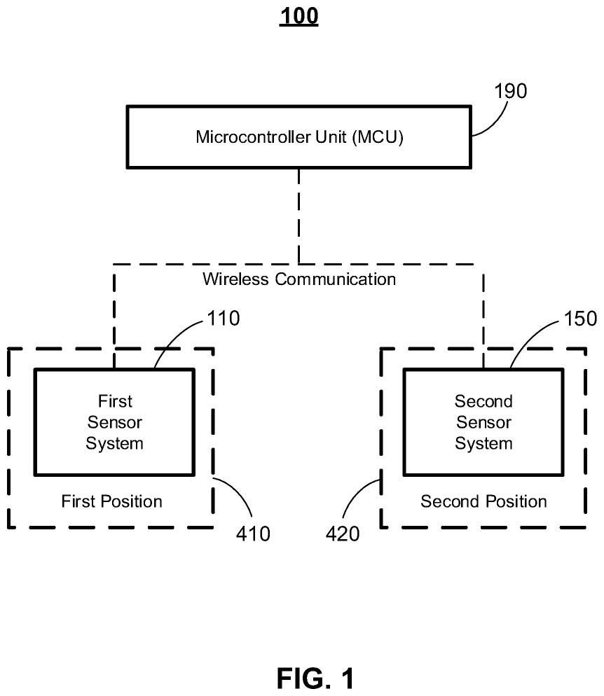

In one aspect, the invention relates to an apparatus for measuring physiological parameters of a mammal subject. Specifically, the measuring can be non-invasive and continuous. FIG. 1 schematically shows a functional block diagram of an apparatus according to certain embodiments of the present invention. It should be noted that the apparatus as shown in FIG. 1 is an exemplary apparatus, and is not intended to limit the apparatus for measuring physiological parameters of a mammal subject.

As shown in FIG. 1, the exemplary apparatus 100 includes a plurality of sensor systems 110 and 150, namely a first sensor system 110 and a second sensor system 150, and a microcontroller unit (MCU) 190 adapted in wireless communication with the sensor systems 110 and 150. The sensor systems 110 and 150 are time-synchronized and communicate with each other wirelessly and bidirectionally, and are respectively attached to the mammal subject. In certain embodiments, the mammal subject can be a human subject or a non-human subject. In certain embodiments, each of the sensor systems is an epidermal electronic system (EES). For example, FIG. 1 shows that the first sensor system 110 is attached to a first position 410 of the mammal subject for detecting a first signal of the mammal subject, and the second sensor system 150 is attached to a second position 420 of the mammal subject for detecting a second signal of the mammal subject. In certain embodiments, the second position 420 is more distal or proximal to a heart of the mammal subject than the first position 410. For example, in one exemplary embodiment, the first position 410 is located at a torso region of the mammal subject, and the second position 420 is located at an extremity region or a limb region of the mammal subject. In this case, the first signal may be a heartbeat signal measured from the torso region, and the second signal may be a pulse signal measured from the extremity region or the limb region. In other words, the first sensor system 110 is a torso sensor system, and the second sensor system 150 is a limb sensor system. In other embodiments, the first position 410 and the second position 420 may be located at different regions of the mammal subject, as long as the first position 410 and the second position 420 are spatially separated. In certain embodiments, the first sensor system 110 can be an electrocardiography (ECG) sensor system, and the second sensor system 120 can be a photoplethysmography (PPG) sensor system. In certain embodiments, the first sensor system 110 and the second sensor system 150 can be implemented as separate physical devices. Alternatively, in certain embodiments, the first sensor system 110 and the second sensor system 150 can reside in a single physical device integrally.

Each of the sensor systems 110 and 150 includes one or more sensors that are used to detect a vital sign of the mammal subject, and then to generate one or more corresponding physiological parameters. Each of the sensor systems 110 and 150 also includes a flexible PCB (fPCB), on which the one or more sensors are disposed. In certain embodiments, the sensors may be various types of sensors for detecting the vital sign as a signal, and the signal can be, for example, an electrical signal related to at least one of electrocardiography (ECG) and electromyography (EMG) technology; a mechanical signal related to movement, respiration and arterial tonometry; an acoustic signal related to vocal cord vocalization and heart sound; and an optical signal related to at least one of blood oxygenation and blood pressure. The MCU 190 is configured to receive, from the sensor systems 110 and 150, output signals representing the physiological parameters, and to display the physiological parameters of the mammal subject. In certain embodiments, the MCU 190 may further process the output signals to obtain a specific vital sign of the mammal subject.

In certain embodiments, the apparatus 100 as shown in FIG. 1 may be in the form of a mobile or wearable device or platform, which increases the flexibility of the apparatus.

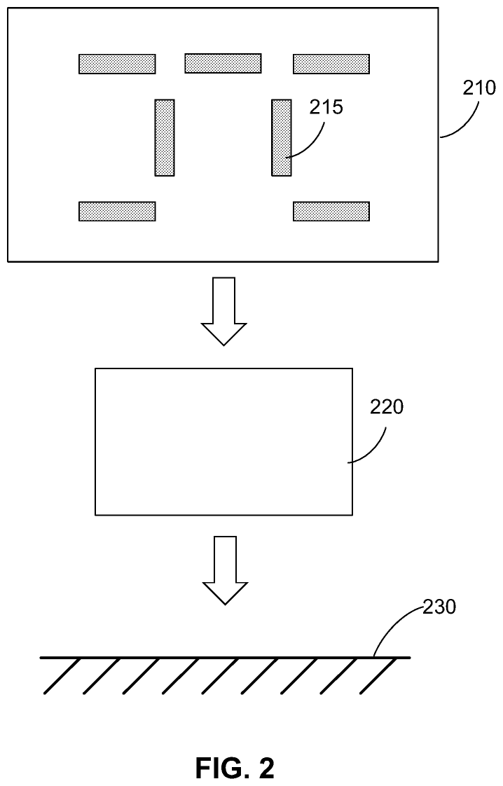

FIG. 2 schematically shows a sensor system being attached to the skin of a mammal subject by the adhesive layer according to certain embodiments of the present invention. As shown in FIG. 2, each sensor system 210 is correspondingly provided with an adhesive layer 220, and the adhesive layer 220 is correspondingly disposed between the sensor system 210 and the skin 230 of the mammal subject. In other words, the apparatus includes a plurality of adhesive layers 220 corresponding to the sensor systems 210, such that each sensor system 210 may be respectively attached to the skin 230 of the mammal subject through the corresponding adhesive layer 220 at a different location. Specifically, the adhesive layer 220 is switchable chemically or physically between an adhesive state and a non-adhesive state. In this way, when the corresponding adhesive layer 220 is in the adhesive state, it may be used to attach the corresponding sensor system 210 to the mammal subject. On the other hand, when the corresponding adhesive layer 220 is switched chemically or physically to the non-adhesive state, it may allow removal of the corresponding sensor system 210 from the skin 230 of the mammal subject.

The adhesive layer 220 separates the sensor system 210 from the underlying skin 230 of the mammal subject. In certain embodiments, the adhesive layer 220 may be a hydrogel adhesive. Hydrogels offer unique properties as a medical adhesive—low allergenicity, biocompatability, and adaptable adhesive strength. Since hydrogel adhesives can either be physically or chemically cross-linked, there is an opportunity to reverse the adhesive property of the hydrogels. In some other embodiments, hydrocolloid adhesives, a polymeric film, fiber adhesives, or an acrylic adhesive may also be used as the material of the adhesive layer 220.

Further, as shown in FIG. 2, the sensor system 210 includes a plurality of perforations 215, such that when the sensor system 210 is attached to the skin 230 of the mammal subject through the corresponding adhesive layer 220, the perforations 215 may enable direct access to the corresponding adhesive layer 220 without the need to remove the sensor system 210 from the skin 230. In this case, the perforations 215 allows for direct visualization of underlying skin health without the need for removal of the sensor system 210. This is necessary to evaluate skin irritation, skin infection, or skin keratinization. Further, the perforations 215 allows for access to the underlying adhesive layer 220 with the ability to introduce a chemical or physical means to reduce adhesion strength of the adhesive layer 220 in the removal process. Moreover, the perforations 215 may be provided to avoid areas of skin wounds, open skins or skin rashes, and to reduce the overall weight of the sensor system 210.

In certain embodiments, the quantity, size, shape, location and configuration of the perforations 215 on the sensor system 210 can be specifically designed to improve overall mechanical compliance in tension, bending and twisting of the sensor system. In certain embodiments, the design of the perforations 215 allows for novel configurations that reduce the peel force when the sensor system 210 is removed along a specific axis, and allows for controlled deformations across specific axes to allow for predictable mechanical deformations and increased mechanical robustness.

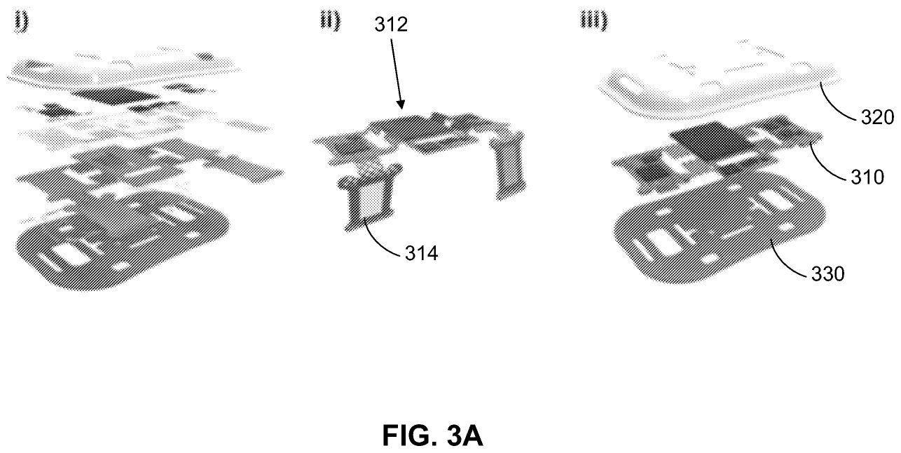

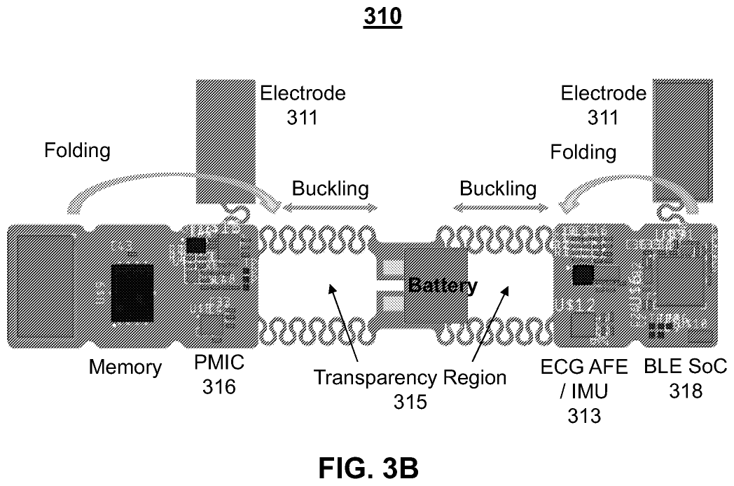

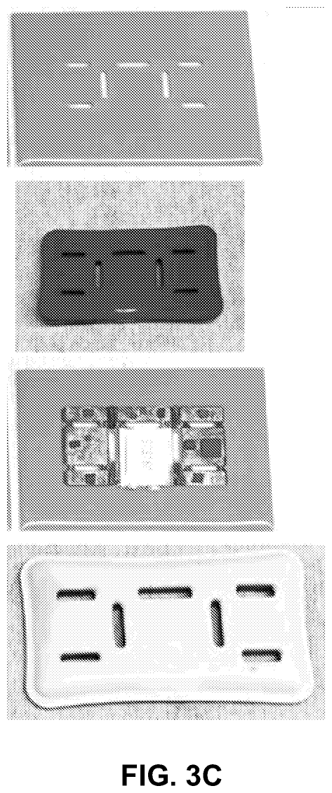

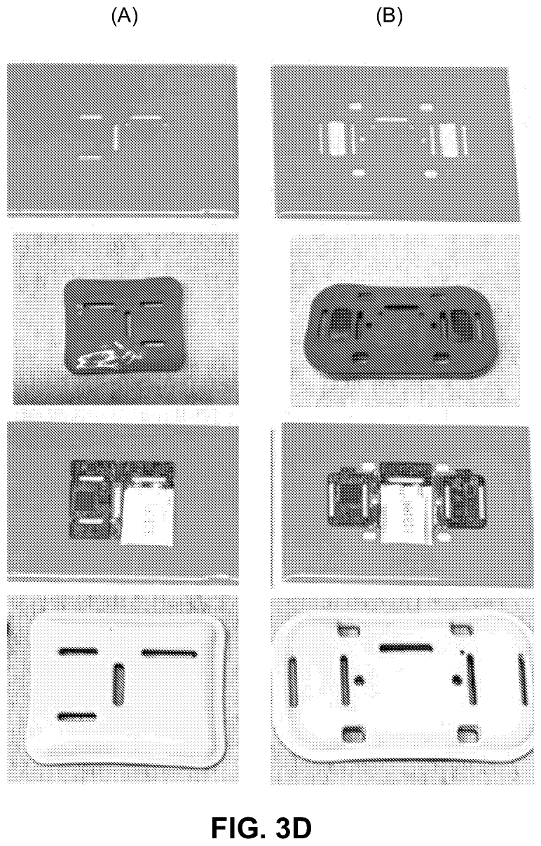

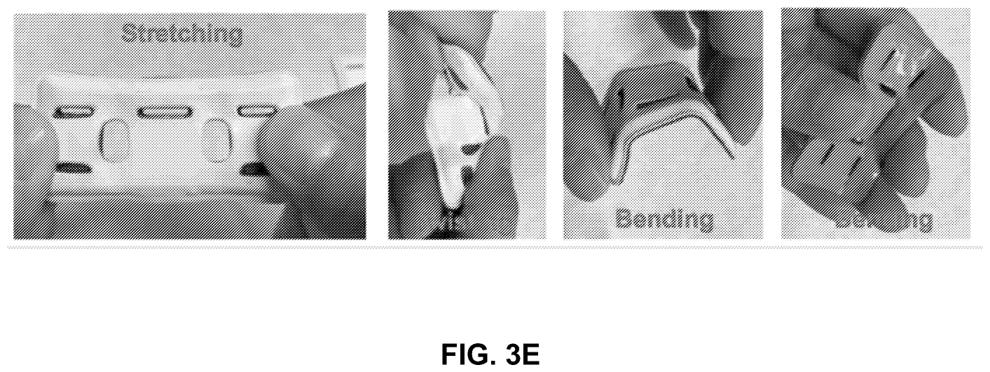

The ability to create the sensor systems with perforations require integrated design of the components of the sensor systems. For example, FIG. 3A schematically shows (i) an explosive view of a sensor system, (ii) a middle circuit layer of the sensor system, and (iii) an explosive view of the layers of the sensor system according to certain embodiments of the present invention. As shown in FIG. 3A, the sensor system includes a middle circuit board layer 310 sandwiched by a top elastomeric encapsulation layer 320 and a bottom elastomeric encapsulation layer 330. Specifically, the middle circuit board layer 310 may be formed by one or more foldable electronic boards, which includes a plurality of electronic components 312 and a plurality of flexible and stretchable interconnects 314 disposed thereon. The flexible and stretchable interconnects 314 are electrically connected to different electronic components 312. The bottom elastomeric encapsulation layer 330 forms a tissue-facing surface attached to the mammal subject, and the top elastomeric encapsulation layer 320 forms an environment-facing surface.

FIG. 3B schematically shows an exemplary middle circuit layer of the sensor system according to certain embodiments of the present invention. As shown in FIG. 3B, the electronic components 312 on the middle circuit layer 310 include two electrodes 311, a ECG AFE/IMU 313, a plurality of transparent regions 315 (which may correspond to the perforations), a PMIC 316, and a Bluetooth system on a chip (SoC) 318. Specifically, the middle circuit layer 310 is in the form of a foldable electronic board, which is foldable as shown in FIG. 3B. In certain embodiment, the electronic components 312 may further include a power supply, which may be an embedded power supply or a detachable modular power supply. In one embodiment, the power supply may be a battery.

As discussed above, the quantity, size, shape, location and configuration of the perforations on each sensor system can be specifically designed for different purposes. For example, FIGS. 3C and 3D schematically shows different sensor systems with perforations having different configurations, sizes and locations according to certain embodiments of the present invention. Specifically, in the sensor system as shown in FIG. 3C and each of the sensor systems (A) and (B) as shown in FIG. 3D, the perforations of each sensor system are formed by corresponding perforations formed on each of the top elastomeric encapsulation layer, the middle circuit board layer and the elastomeric encapsulation layer. In particular, the corresponding perforations formed on the middle circuit board layer exists between the electronic components. In other words, the middle circuit board layer must be designed to allow for holes and perforations, which requires strategic placement of different electronic components and the flexible and stretchable interconnects. Further, the corresponding perforations formed on the top elastomeric encapsulation layer and the bottom elastomeric encapsulation layer are correspondingly designed to integrate and align with the corresponding perforations formed on the middle circuit board layer. Thus, all corresponding perforations on different layers align with each other to form the perforations of the sensor system.

FIG. 3E schematically shows deformation of the layers of the sensor system according to certain embodiments of the present invention. As shown in FIG. 3E, each of the layers of the sensor system may be subject to stretching, twisting and/or bending to a certain degree in different directions, thus providing greater mechanical compliance with specific deformations.

As discussed above, the adhesive layer (e.g., hydrogel adhesives) can either be physically or chemically cross-linked, such that there is an opportunity to reverse the adhesive property thereof. This feature may be accomplished via a number of different strategies. For example, in certain embodiment, the adhesive layer may be switchable chemically from the adhesive state to the non-adhesive state by applying a liquid or a chemical solution directly to the adhesive layers through the perforations. Specifically, this chemical means of the adhesive layer may be achieved by:
application of water via a syringe from the top surface directly to the underlying adhesive would lead to a natural swelling of the hydrogel and lose of adhesive strength
application of normal saline via a syringe from the top surface directly to the underlying adhesive would lead to a natural swelling of the hydrogel and lose of adhesive strength
other reactions include thiol-disulfide exchange reactions, retro-Michael reactions, or retro Diels-Alder reactions for dissolvable hydrogel adhesives
solutions of varying levels of pH
glucose solutions can be used to dissolve glucose-sensitive hydrogels

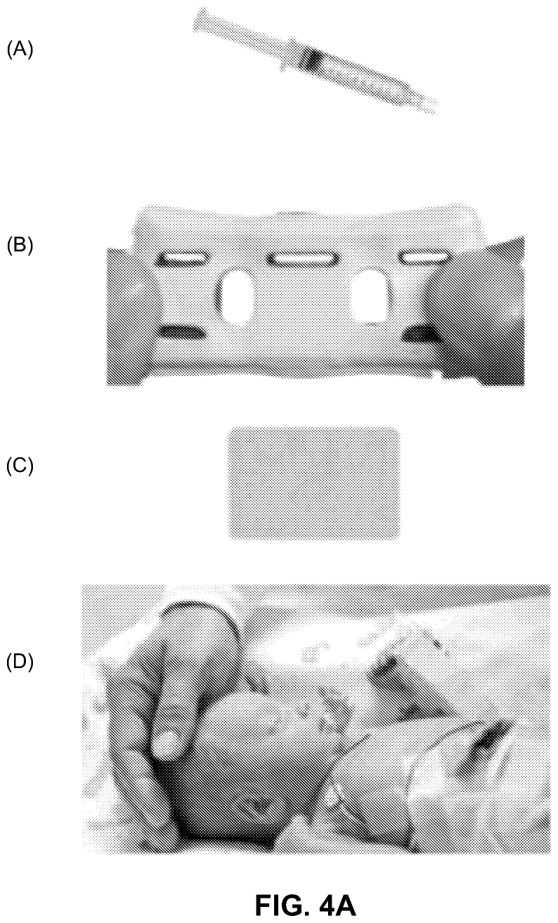

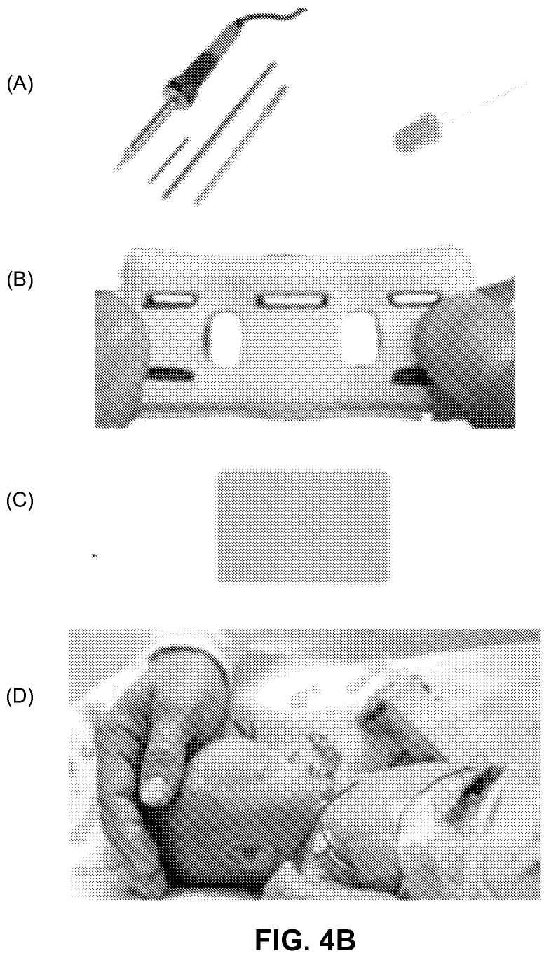

FIG. 4A schematically shows switching an adhesive layer chemically from the adhesive state to the non-adhesive state according to certain embodiments of the present invention. As shown in FIG. 4A, a syringe provided with the liquid or the chemical solution is provided to perform the chemical dissolution of the adhesive layer in step (A). Since the sensor system is provided with perforations as shown in step (B), the liquid or the chemical solution may be applied directly to the adhesive layer through the perforations. In certain embodiments, the liquid or the chemical solution may be water, normal saline, or other types of liquid or chemical solutions.

Once the liquid or the chemical solution is applied, the adhesive layer as shown in step (C) may completely or partially dissolve underneath the sensor system, thus reducing the adhesiveness of the adhesive layer (e.g., via natural swelling of the hydrogel). In this case, the peel force may drastically diminish, allowing easy removal of the sensor system from the skin of the mammal subject in step (D).

In certain embodiment, the adhesive layer may be switchable physically from the adhesive state to the non-adhesive state through heat or light. Specifically, this physically means of the adhesive layer may be achieved by:
heat (temperature sensitive physically cross linked hydrogels would be dissolved in this manner)
light

FIG. 4B schematically shows switching an adhesive layer physically from the adhesive state to the non-adhesive state according to certain embodiments of the present invention. As shown in FIG. 4B, a heating device or a light source is provided to perform the physical dissolution of the adhesive layer in step (A). Since the sensor system is provided with perforations as shown in step (B), the light may be applied directly to the adhesive layer through the perforations. Once the heat or light is applied, the adhesive layer as shown in step (C), which is thermally or optically sensitive, may completely or partially dissolve underneath the sensor system, thus reducing the adhesiveness of the adhesive layer. In this case, the peel force may drastically diminish, allowing easy removal of the sensor system from the skin of the mammal subject in step (D).

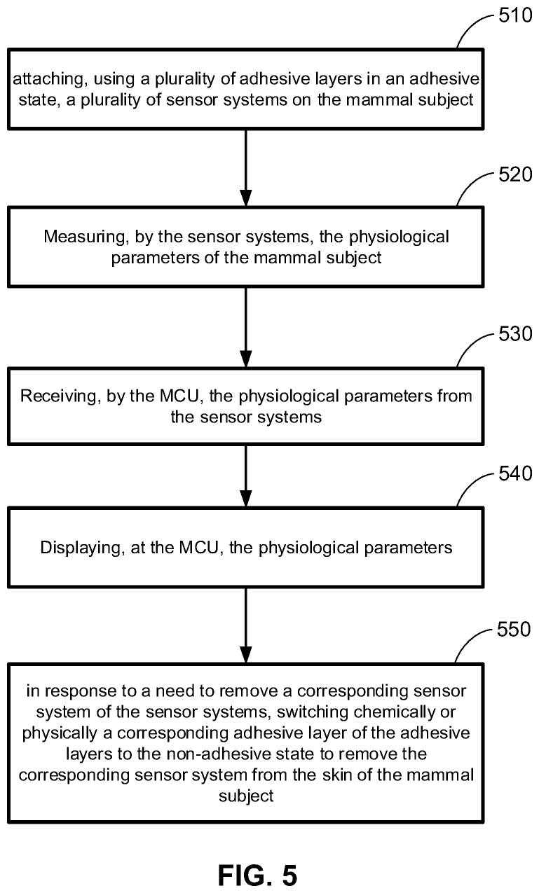

FIG. 5 shows a flowchart of a method of measuring physiological parameters of a mammal subject according to certain embodiments of the present invention. In certain embodiments, the method as shown in FIG. 5 may be implemented on the apparatus as shown in FIG. 1 and the sensor system as shown in FIG. 2. It should be particularly noted that, unless otherwise stated in the disclosure, the steps of the method may be arranged in a different sequential order, and are thus not limited to the sequential order as shown in FIG. 5.

As shown in FIG. 5, at procedure 510, the sensor systems (i.e., the first sensor system 110 and the second sensor system 150 as shown in FIG. 1) are attached on the skin of the mammal subject using a plurality of adhesive layers 220 as shown in FIG. 2. For example, the first sensor system 110 is attached to a first position in the torso region 410 of the mammal subject for measuring a heartbeat of the mammal subject, and the second sensor system 150 is attached to a second position in the limb region 420 of the mammal subject for measuring a pulse of the mammal subject. Further, the sensor systems 110 and 150 are in wireless communication with the MCU 190, and are time-synchronized and spatially separated by a distance defined by the first and second positions. Each adhesive layer is switchable chemically or physically between an adhesive state and a non-adhesive state, and in this case, the adhesive layer is in the adhesive state such that the sensor systems may be attached on the skin of the mammal subject.

At procedure 520, the sensor systems 110 and 150 are used to measure or monitor the physiological parameters of the mammal subject. In certain embodiments, the physiological parameters of the mammal subject may include one or more of: heart rate, heart rate variability, heart sounds, blood pressure, chest wall displacement, electromyography, electrocardiography, blood oxygenation, respiratory rate, respiratory effort, respiratory cadence, tidal volume, coughing, snoring, sneezing, throat clearing, wheezing, apnea, hypoapnea, physical activity, core body position, peripheral limb position, scratching, vocalizations, rubbing, walking, sleep quality, sleep time, wake time upon sleeping, skin temperature, core body temperature, and a combination thereof. Once the physiological parameters are obtained, the sensor systems 110 and 150 may respectively generate corresponding output signals, which are then transmitted wirelessly to the MCU 190.

At procedure 530, the MCU 190 receives the physiological parameters from the sensor systems 110 and 150. Specifically, the MCU 190 receives the output signals from the first sensor system 110 and the second sensor system 150, and then processes the output signals to obtain the physiological parameters. At procedure 540, the MCU 190 may display the physiological parameters.

At procedure 550, whenever there is a need to remove a corresponding sensor system, a user may, in response to the need, switch (chemically or physically) the corresponding adhesive layer to the non-adhesive state to remove the corresponding sensor system from the skin of the mammal subject. Examples of switching the adhesive layer by chemical and physical means may be shown in FIGS. 4A and 4B, respectively.

In a further aspect, the invention relates to a non-transitory tangible computer-readable medium storing instructions which, when executed by one or more processors, cause the methods as discussed above to be performed.

Demonstrations on adult volunteer subjects and patients in an operating NICU illustrate the operational features in realistic settings.

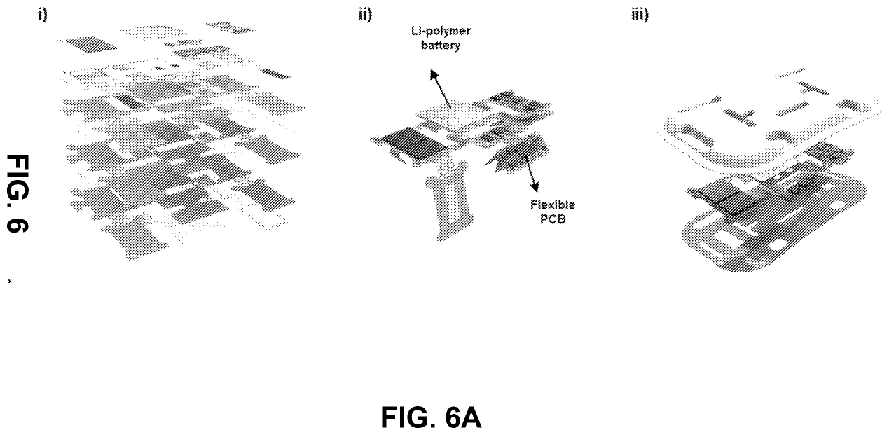

Thin, soft, perforated platform for wireless health monitoring in the NICU/PICU FIGS. 6A and 6B shows schematic diagrams and images of a soft, perforated wireless device for measuring electrocardiograms (ECGs) and skin temperature, and for capturing tri-axis accelerometry data according to certain embodiments of the present invention. Specifically, FIG. 6A shows an exploded view illustration of a device with a rechargeable battery. The 4-layer flexible printed circuit board supports circuit components on island structures, with serpentine filamentary interconnects configured for a folding process conducted prior to encapsulation. Precompression of these interconnects increases the elastic stretchability of the system. Openings formed through the elastomeric encapsulation structure provide key advantages in mechanics, adhesion release, visual inspection and breathability. FIG. 6B shows (a) a block diagram of the operational scheme of the device with analog-front-end for ECG processing, tri-axis accelerometer, thermometer IC, and BLE SoC, and (b) shows images of a device on the chest of a realistic model of a neonate.

As shown in FIG. 6A, the electronics consist of a flexible printed circuit board (fPCB; total thickness 173 μm) that includes four layers of copper (Cu) and five layers of polyimide (PI) structured to include thin, narrow serpentine conductive traces (widths between 76 and 375 μm wide; thickness 18 μm) that interconnect a collection of chip-scale integrated components (lateral dimensions between 1 mm² and 64 mm²; thicknesses between 0.5 mm and 1.5 mm). A small, rechargeable lithium-polymer battery (DNK201515; 15 mm×15 mm; thickness 2 mm) supplies power for operation. As described in detail in subsequent sections, holes formed through the fPCB and the encapsulation structure reduce the effective modulus and bending rigidity to facilitate gentle application and removal from the skin. Additional enhancements include layouts with predefined, molded curvature designed to match that of a targeted body location. The system supports wireless monitoring of the full range of vital signs and other parameters described in the introduction, using Bluetooth protocols to allow the use of a standard smartphone as the graphical user interface and location for data storage and processing.

As shown in FIG. 6B(a), these functions follow from an associated collection of sensors that includes an inertial measurement unit (LSM6DSL, STMicroelectronics), a clinical-grade temperature gauge (MAX30205, Maxim Integrated), and a sub-system for recording electrocardiograms (ECGs) through two gold-plated electrodes and a biopotential analog front-end (MAX30001, Maxim Integrated). A Bluetooth Low Energy (BLE) system-on-a-chip (SoC) supports wireless transfer of data acquired from these sensors for real-time display on a tablet computer and/or for storage on a memory module including in the device itself (MT29F2G01ABAGDWB, Micron Technology Inc.). The battery has a storage capacity of 20mAh, sufficient to support operation for 24 hours before requiring a recharge. A power management circuit includes a regulator to convert the voltage (3.7 V) of the battery to the voltage (3.3 V or 1.8 V) necessary for the components of the device. A wireless circuit uses an induction coil for charging the battery.

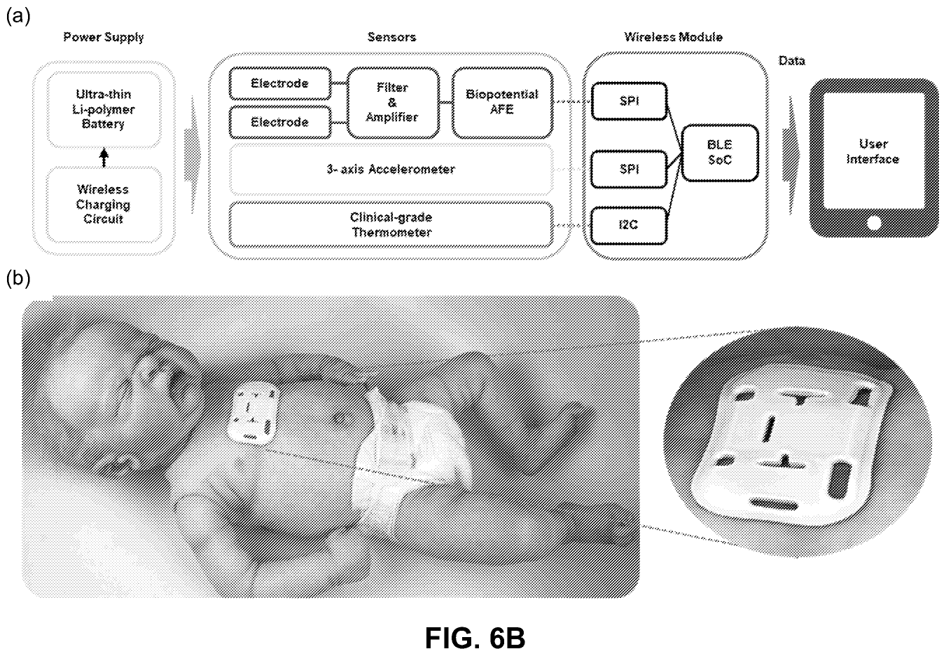

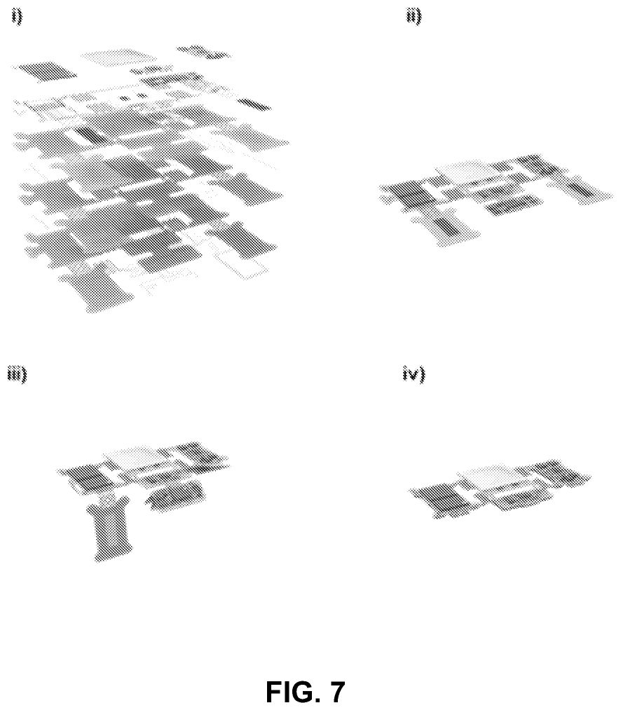

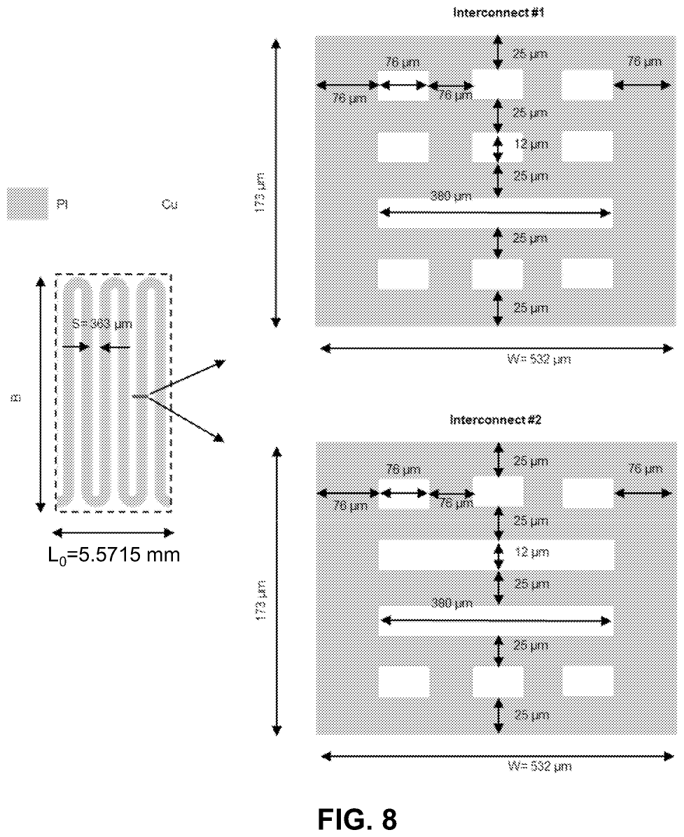

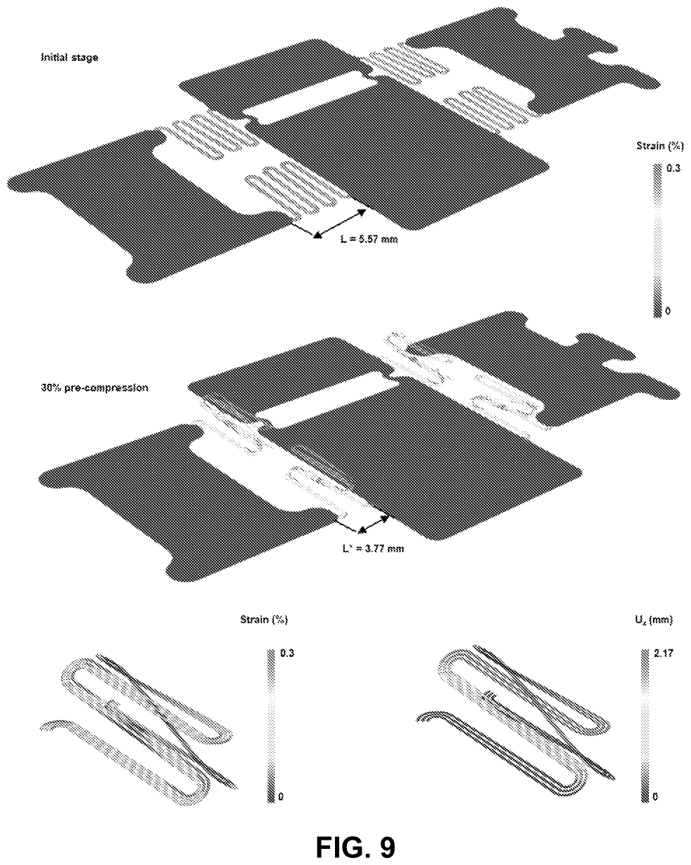

The assembly and encapsulation process begins with folding of the fPCB islands at hinge points defined by serpentine interconnects, to reduce the overall size of the device (FIG. 6a, middle and FIG. 7). Specifically, FIG. 7 schematically shows the design and construction of a holey device platform according to certain embodiments of the present invention. As shown in FIG. 7, after the 4-layer flexible printed circuit board is assembled with components and a Li-polymer battery, the sub-islands are folded and the serpentines between three main islands are pre-compressed. The serpentines include four Cu layers, pre-compressed by 30% to enhance their flexibility and stretchability (FIGS. 8 and 9). Specifically, FIG. 8 schematically shows the serpentine interconnects used in a holey device according to certain embodiments of the present invention, and FIG. 9 schematically shows the results of modeling of the mechanical properties of serpentine interconnects for pre-compression according to certain embodiments of the present invention. As shown in FIG. 8, the main islands are connected mechanically and electrically by serpentine interconnects that provide high stretchability and flexibility. These interconnects consist of four 12 μm-thick copper layers with cover layers of polyimide (PI) and separated by 25 μm in the out-of-plane direction. Each copper layer features one trace with a width of 380 μm or three traces with a width of 76 μm and the in-plane distance between the traces is 76 μm. The total thickness of the interconnects is 173 μm. As shown in FIG. 9, the initial lengths of the serpentine interconnects between the three main islands are L=5.57 mm. To increase the elastic stretchability, the interconnect is pre-compressed such that its initial horizontal length is reduced from L=5.57 mm to L*=3.37 mm. For 30% compression, the strain in the top copper layer exceeds the elastic limit (0.3%) but remains below the fracture strain <1%. The maximum vertical displacement of the serpentine interconnect is 2.17 mm.

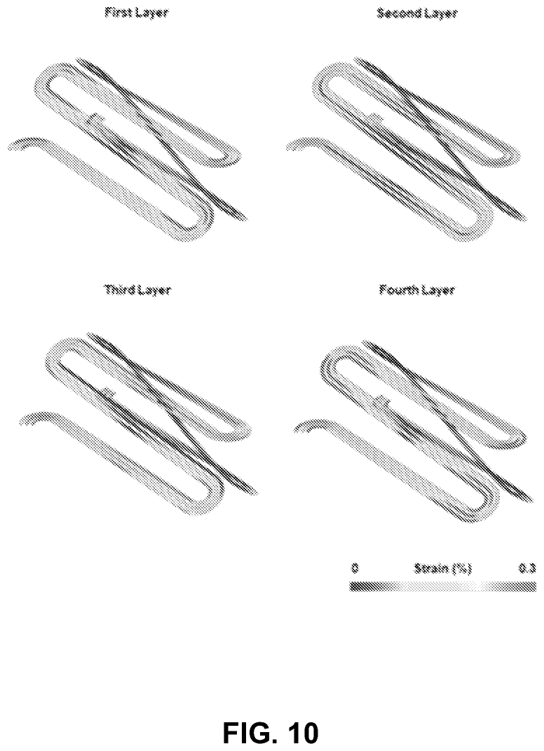

FEA results indicate that the strain in each Cu layer remains below the fracture limit (˜1%) throughout these steps (FIGS. 8, 9 and 10). Specifically, FIG. 10 schematically shows the computed distributions of strain in the copper layers of the serpentine interconnects associated with pre-compression according to certain embodiments of the present invention. As shown in FIG. 10, for 30% compression, the strain in the first and fourth copper layers exceed the elastic limit (0.3%) but remains below the fracture strain <1% while the strain in the second and third layer remains below the elastic limit.

After this assembly process, the serpentine interconnects can stretch along their length by up to 85% without inducing fractures in the Cu (FIG. 11). Specifically, FIG. 11 schematically shows the modeling of the strain distributions in the copper layers of the serpentine interconnects for stretching deformations according to certain embodiments of the present invention. As shown in FIG. 11, for 85% uniaxial stretching, the strains in the first and fourth copper layer exceed the elastic limit (0.3%) but remain below the fracture strain <1% while the strains in the second and third layer remain below the elastic limit.

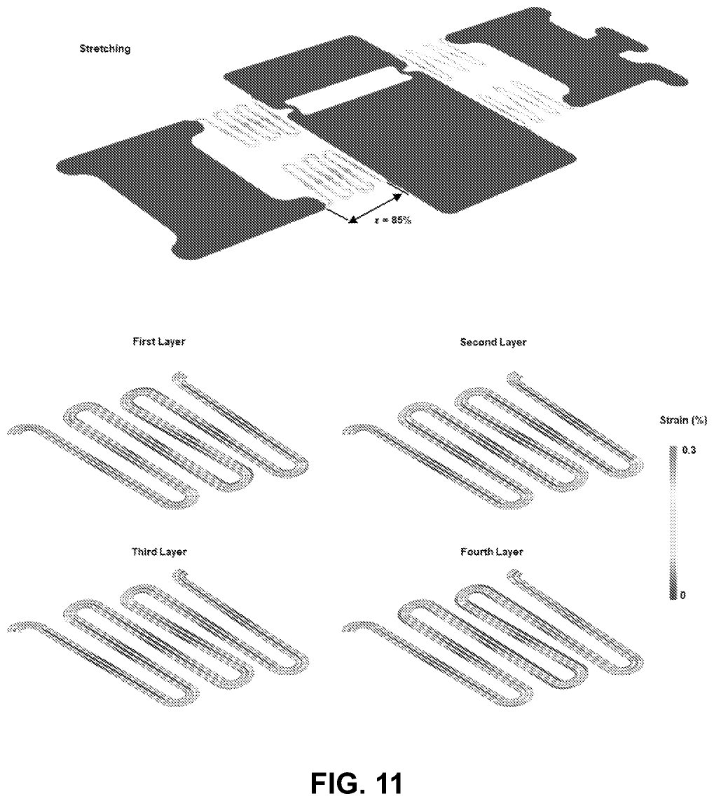

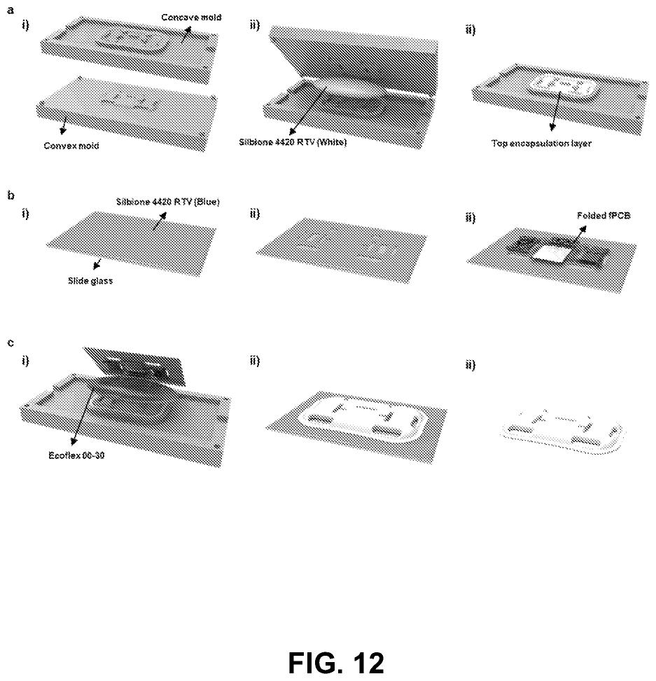

The top and bottom layers of the encapsulation structure consist of a low modulus silicone material (5 MPa; Silbione RTV 4420) molded into the desired shapes. A silicone polymer with a different formulation (0.069 MPa; Ecoflex 00-30) injected in between these layers fills the empty spaces in a manner that maintains the softness of the overall platform and minimizes mechanical constraints on motions of the serpentine interconnects induced by bending or stretching. This material also prevents damage of the encapsulation layer by the rigid electronic components and entanglement of the serpentine interconnects (FIG. 6a, right and FIG. 12). Specifically, FIG. 12 schematically shows a summary of the process for encapsulation of a holey device according to certain embodiments of the present invention. As shown in FIG. 12a, i) the concave and convex aluminum molds for the top encapsulation layer. Then, ii) using a pair of molds, silbione RTV 4420 with white color dye is pressed and cured in between. Subsequently, iii) a thin top encapsulation layer with holes, with a thickness of 300 μm. As shown in FIG. 12b, i) a flat film of silbione RTV 4420 with blue dye spin-coated on a slide glass as a bottom encapsulation layer.

Then, ii) laser cutting defines openings for two ECG electrodes and for the holes. Subsequently, iii) two ECG electrodes on the folded electronic platform attach to the bottom encapsulation layer using an adhesive. As shown in FIG. 12c, i) a top encapsulation with concave mold and bottom layer with electronics are assembled with uncured silicone elastomer (Ecoflex 0-30) as an inner material. Then, ii) a fully encapsulated holey device is obtained. Subsequently, iii) the edges are cut, and the device is released from slide glass.

The photograph in FIG. 6c shows a device with this construction mounted on the curved chest of a realistic neonate model, bonded to the surface with a hydrogel adhesive (KM 40A hydrogel, Katecho Inc.; thickness ˜815 μm).

Mechanical Characteristics Associated with Holey Designs

Some holes reside near the serpentine interconnects to preserve their high flexibility and stretchability; others lie inside the center island to facilitate deformations in this part of the device. Holes of various shapes near the border area promote low stiffness and modulus at locations where peeling initiates, to reduce skin irritation during device application and removal. Systematic studies by FEA define various advantages of this layout in effective modulus and bending stiffness, as well as in corresponding degrees of stretchability and bendability.

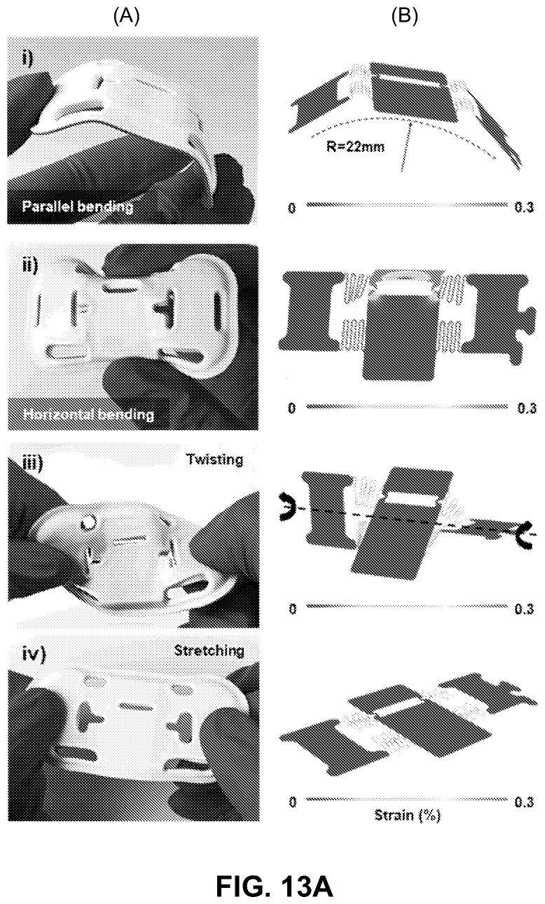

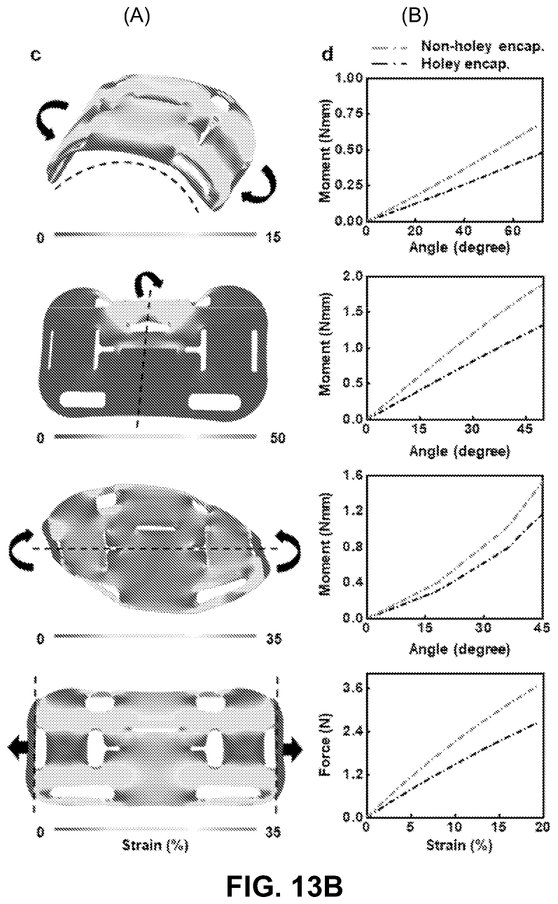

FIGS. 13A and 13B schematically show the mechanical characterization results and images of a soft, holey, wireless vital signs monitoring device under various mechanical deformations according to certain embodiments of the present invention. Specifically, FIG. 13A shows (A) images of a representative device during (i) parallel bending, (ii) horizontal bending, (iii) twisting and (iv) stretching; and (B) simulation results for the deformed geometries and strain distributions in the copper layer of the electronic system. FIG. 13B shows (A) simulation results for the deformed geometries and strain distributions in entire encapsulated device during corresponding deformations; and (B) comparisons of moment-angle and force-strain responses for holey and non-holey device designs.

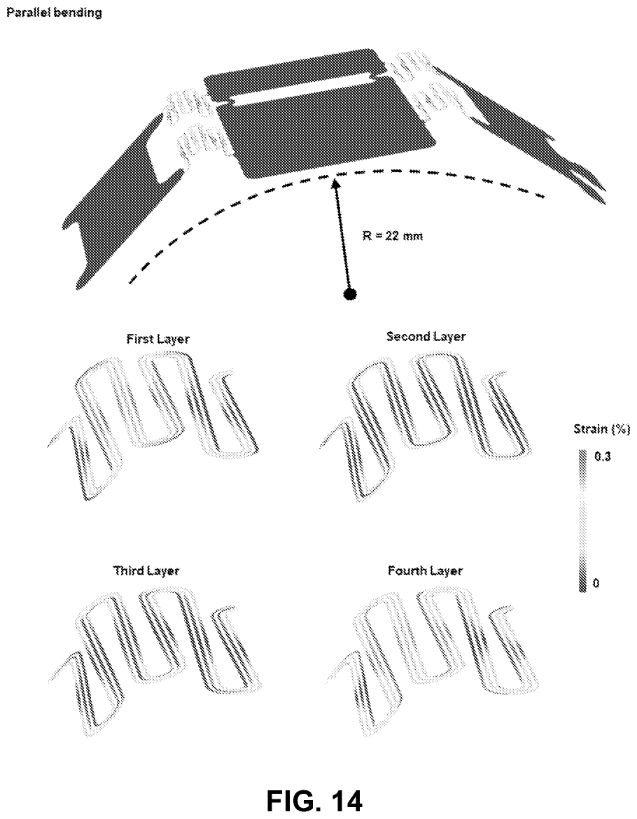

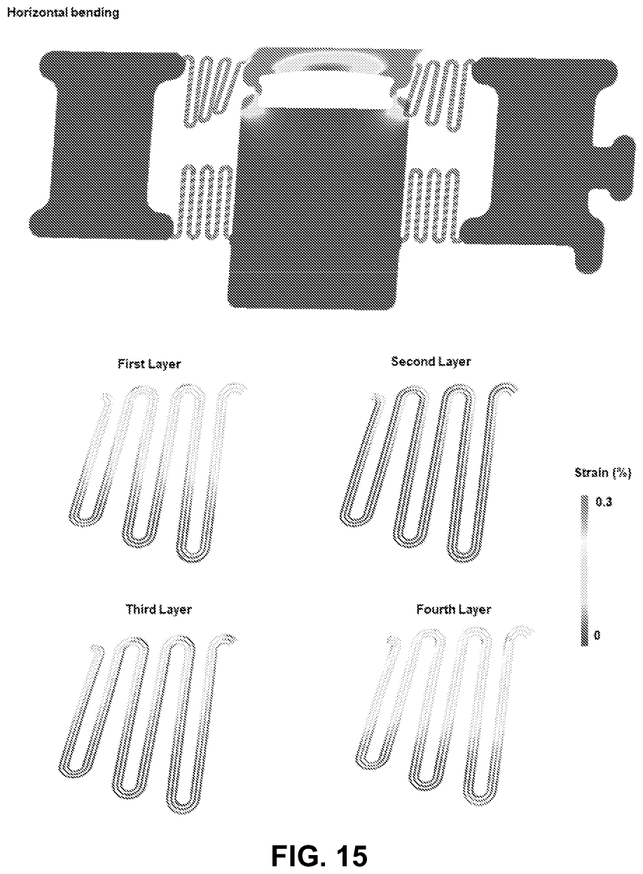

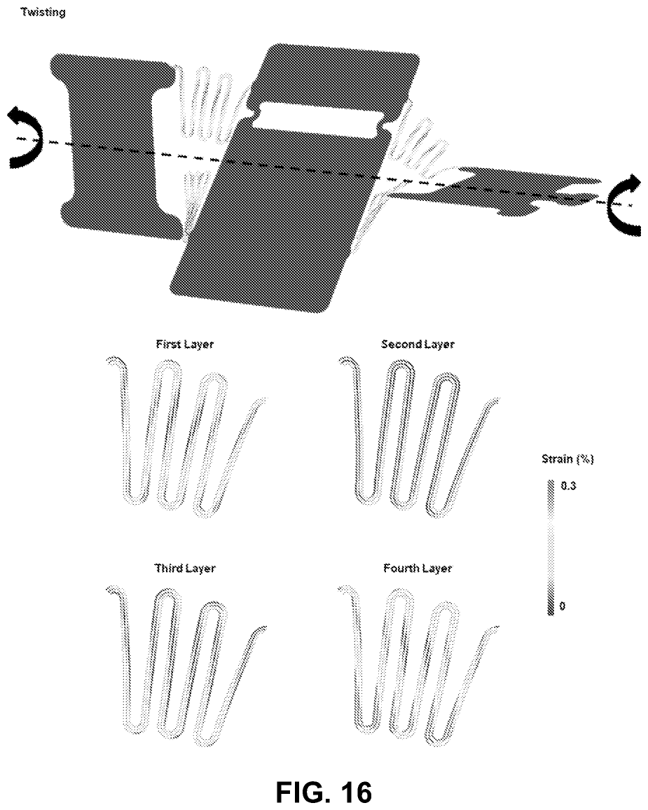

The photograph images in FIG. 13A show the device bent along its long axis to a radius of 22 mm (left) and along the orthogonal direction to a radius of 5.3 mm (middle) and through a twisting angle of 125° (right). The three main islands that support the IC chips and the battery include stiffeners of rigid printed circuit board material (Garolite G-10/FR-4, thickness 381 μm). These elements effectively eliminate bending in these areas, to avoid possible damage to the solder joints between the components and the fPCB. All system level deformations of the device involve motions of the serpentine interconnects, such that strains in the Cu ultimately limit the range of stretchability and bendability. The maximum principal strains in the Cu layers remain below the yield strain of 0.3% (FIG. 13A(B) and FIGS. 14-16), for the cases examined here. Specifically, FIG. 14 schematically shows the computed strain distribution in the copper layers of the serpentine interconnect for bending deformations in the parallel direction according to certain embodiments of the present invention. As shown in FIG. 14, for a bending radius of 22 mm, the strains in all the copper layers remain below the elastic limit (0.3%). FIG. 15 schematically shows the computed strain distribution in the copper layers of the serpentine interconnect for bending deformations in the horizontal direction according to certain embodiments of the present invention. As shown in FIG. 15, for a bending radius of 5.3 mm, the strains in all of the copper layers remain below the elastic limit (0.3%). The connecting islands that support the electronics components are assumed to be significantly stiffer than the serpentines. FIG. 16 schematically shows the computed strain distributions in the copper layers of the serpentine interconnect for twisting deformations according to certain embodiments of the present invention. As shown in FIG. 16, for a twisting angle of 125°, the strains in the first and fourth copper layers exceed the elastic limit (0.3%) but remain below the fracture strain <1%. The strains in the second and third layer remain below the elastic limit.

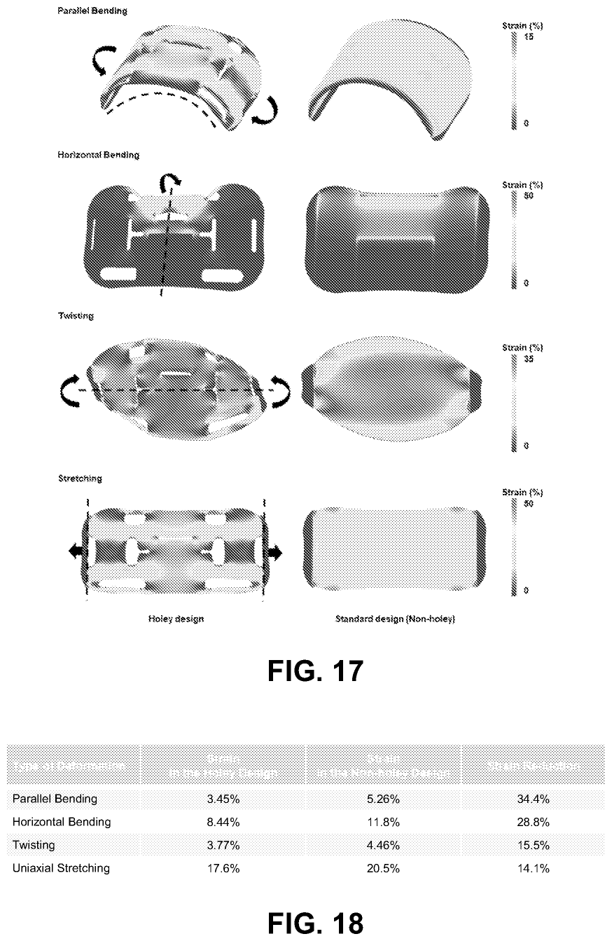

Comparisons to standard (i.e. non-holey) devices with otherwise similar designs reveal that the presence of the holes reduces the strains across all functional materials by 34.4% (bending), 28.8% (orthogonal bending), 15.5% (twisting), 14.1% (stretching) for these cases (FIG. 13B(A), FIG. 17, and the table in FIG. 18). Specifically, FIG. 17 schematically shows comparisons of computed strain distributions in the encapsulated device with and without holes under various deformations. As shown in FIG. 17, the bending radii in the parallel and horizontal direction are 8 mm and 3 mm. The twisting angle is 125° and the stretching is 20%. FIG. 18 shows a table of comparison of computed strain distributions and strain reductions in the encapsulated devices with holey and non-holey designs for various deformations.

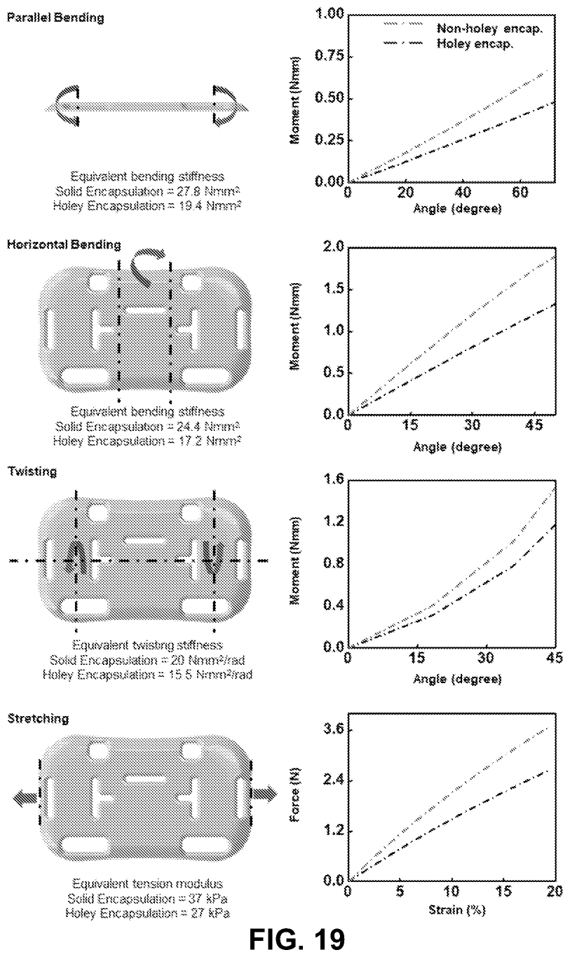

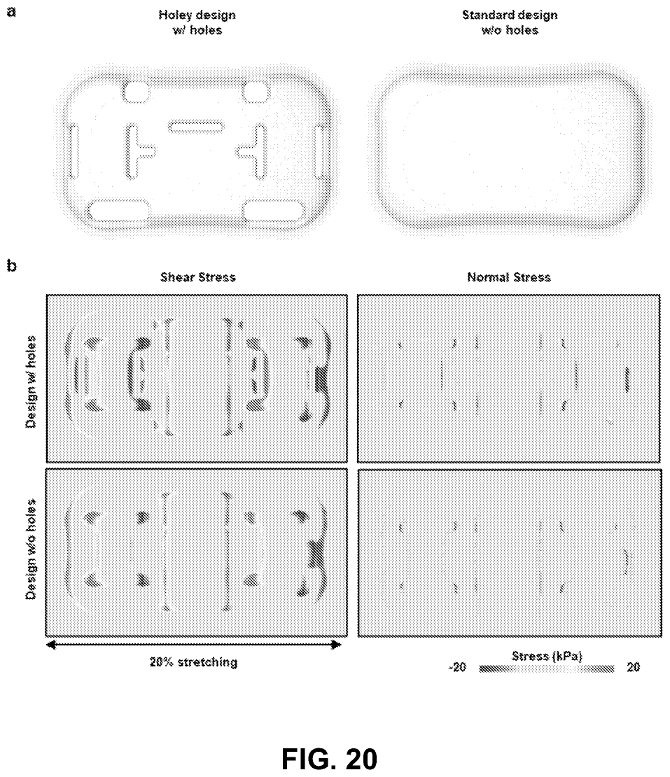

Similar comparisons indicate that the holes decrease the stiffness and modulus by ˜30% for parallel/horizontal bending, ˜23% for twisting and ˜20% for uniaxial stretching (FIG. 13B(B) and FIG. 19). These features also lead to stresses at the interface with the skin that remain below thresholds for sensory perception for adults (20 kPa) for uniaxial stretching of up to 20%, a value at the high end of the range expected in practical use (FIG. 20). Specifically, FIG. 19 schematically shows Comparisons of computed moment-angle and force-strain responses for holey and non-holey device designs for deformations such as parallel bending, horizontal bending, twisting and stretching according to certain embodiments of the present invention. FIG. 20 schematically shows the computed results for shear and normal stress at the interface between holey and non-holey device designs and skin according to certain embodiments of the present invention. Specifically, FIG. 20a shows the illustrations of the holey and non-holey device designs, and FIG. 20b shows the computed shear and normal stresses in the skin are simulated after 20% stretching.

Mechanical Characteristics Associated with Curved Designs

Devices with pre-defined curvature further improve the mechanics at the skin interface. Applying conventional planar devices onto the surfaces of the body requires bending. The associated bending-induced stresses can promote delamination in certain cases and they can cause skin irritation in others. Both effects are pronounced for premature neonates due to their small, highly curved anatomical features and their fragile skin. The mitigation strategy introduced here exploits encapsulation layers formed in curved geometries approximately matched to those of the desired mounting location and size/age of the baby.

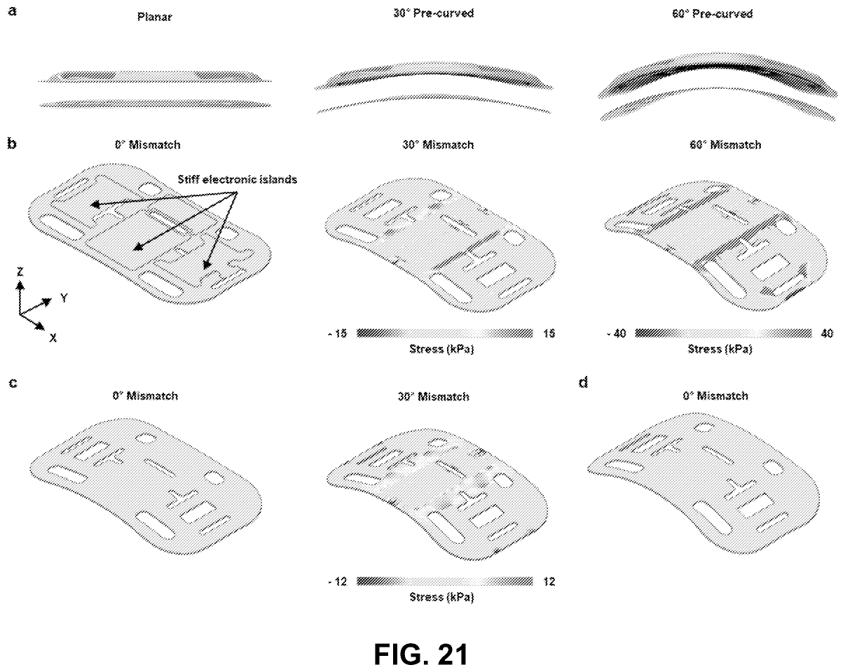

FIG. 21 schematically shows the pre-curved holey ECG devices with different curvatures according to certain embodiments of the present invention. Specifically, FIG. 21a shows images of (left) planar, (middle) 30° and (right) 60° pre-curved designs. Comparison of computed stresses induced by the resilience from curvature mismatch between the skin and the device. FIG. 21b shows the planar device for 0°, 30° and 60° mismatch. FIG. 21c shows the 30° pre-curved device for 30° mismatch. FIG. 21d shows the 60° pre-curved device for 0° mismatch.

FIG. 21a shows devices with planar and with curved shapes. The examples here involve bending radii (angle) of 108 mm) (30° and 54 mm) (60° matched to the chest circumference of pediatric patients (11-12 ages: approximately 65-70 cm) and newborns (30-35 cm). When attaching the device to the surfaces of skin with different curvatures (planar, 30° curved, 60° curved), mismatch with the curvature of device results in a resilience associated with elastic forces directed toward returning the bent device to its original shape. The stresses caused by this resilience appear most prominently near the islands of the device due to their large stiffnesses compared to those of the encapsulation structures (FIG. 21b-d). For the planar device (FIG. 21b), the maximum stress is −15 kPa (compression) and 15 kPa (tension), −28 kPa (compression) and 38kPa (tension) for cases of 30° and 60° mismatch in curvature, respectively. For the cases of devices with pre-curvatures of 30° (FIG. 3c), the maximum stresses are −12 kPa (compression) and 7 kPa (tension) for a curvature mismatch of 30°. All cases where the curvatures are matched (=0° mismatch) exhibit zero stress.

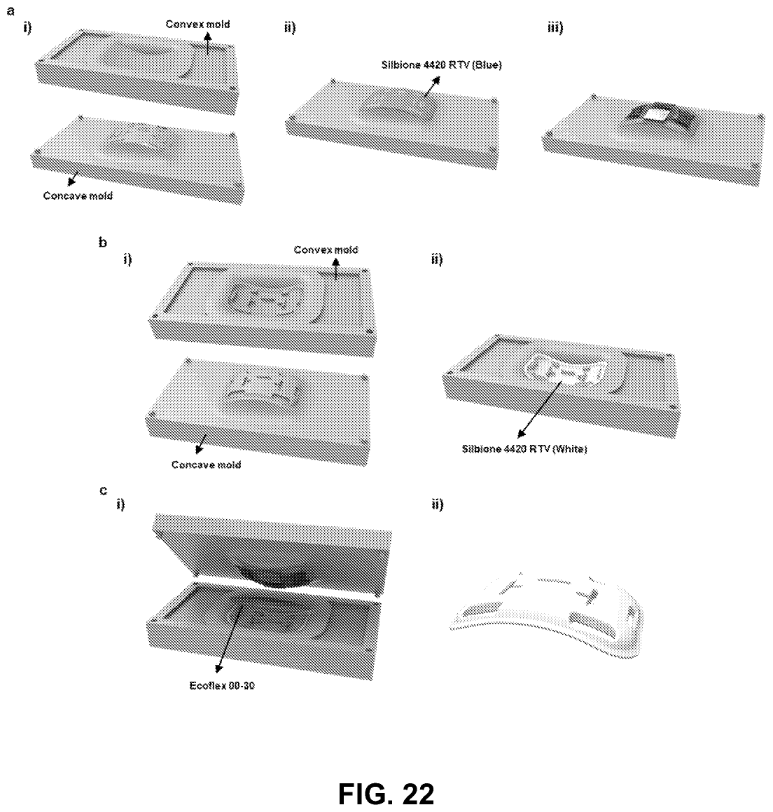

FIG. 22 schematically shows a summary of the process for pre-curved encapsulation of a holey device according to certain embodiments of the present invention. Specifically, FIG. 22a shows i) concave and convex aluminum molds for the pre-curved bottom encapsulation layer. Then, ii) using a pair of molds to form a pre-curved and thin bottom encapsulation layer with holes, with a thickness of 300 μm. Subsequently, iii) two ECG electrodes on the folded electronic platform attach to the pre-curved bottom encapsulation layer using an adhesive. FIG. 22b shows i) concave and convex aluminum molds for the pre-curved top encapsulation layer. Then, ii) using a pair of molds, a pre-curved and thin top encapsulation layer with holes, with a thickness of 300μm. FIG. 22c shows i) a pre-curved top encapsulation (with its concave mold) and a pre-curved bottom layer (with electronics and its convex mold) are assembled with uncured silicone elastomer (Ecoflex 0-30) as an inner material. Then, ii) a fully encapsulated holey device with pre-curved encapsulation layers is obtained.

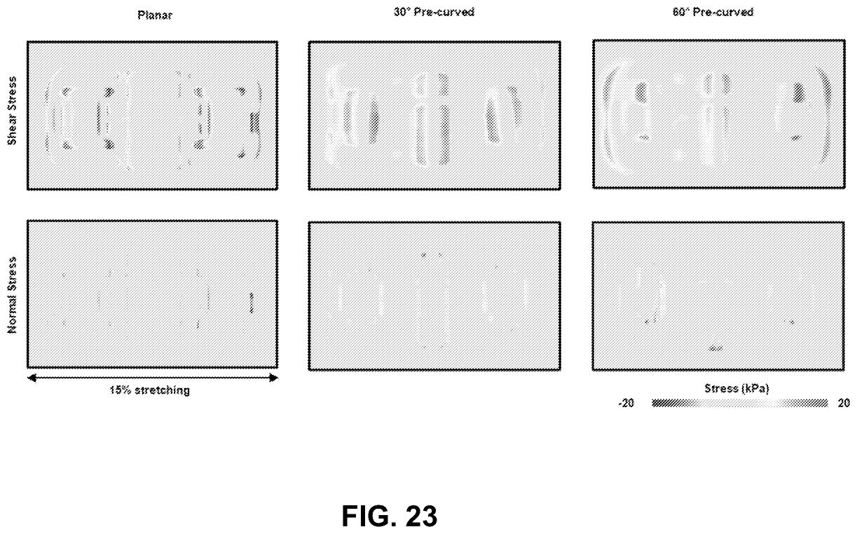

FIG. 23 schematically shows computed shear and normal stress at the interface between skin and holey devices after 15% tangential stretch according to certain embodiments of the present invention. Specifically, FIG. 23 show the calculations of stress distributions for such devices mounted on skin with matching curvature and tangentially stretched to 15%. As shown in FIG. 23, planar, 30° pre-curved, 60° pre-curved holey devices are attached to the skin with matching curvature, respectively. The interfacial stresses are below the somatosensory pressure range (20 kPa) of the skin even when the skin is stretched by 15%. These results of FEA show that the pre-curved encapsulation approach reduces the stress to the skin. These effects minimize mechanically induced irritation to the skin and they promote robust interfaces.

Water-triggered reduction of the strength of adhesion to the skin

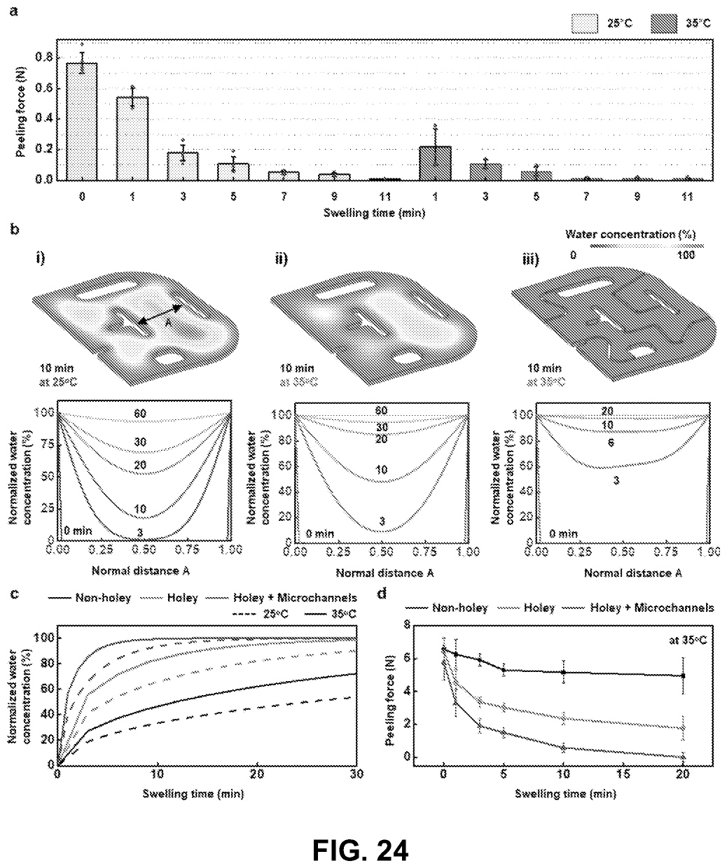

As described previously, the process of removing devices from the skin can cause iatrogenic injuries. Thin, soft and wireless devices significantly reduce the strength of adhesion necessary to support robust bonding compared to that required for conventional wired sensors. Nevertheless, separate, triggering mechanisms to reduce the adhesion to facilitate release can be valuable. In a strategy introduced here, water applied to the holes and the surrounding perimeter of the device cause swelling of a hydrogel bonding layer, thereby reducing the strength of adhesion dramatically, even to levels that can be considered negligible. FIG. 24 schematically shows water triggered soft release of the hydrogel adhesive according to certain embodiments of the present invention. Specifically, FIG. 24a shows the peel force of hydrogel adhesive as a function of swelling time and water temperature (25° C. and 35° C.). FIG. 24b shows computed swelling model of the hydrogel adhesive during water diffusion through the holey device and the difference in water concentration of the hydrogel adhesive as a function of swelling time, water temperature, and adhesive design; (i) holey design at 25° C., (ii) holey design at 35° C. and (iii) holey design with microchannels at 35° C. FIG. 24c shows the comparison of water concentration averaged across the entire volumes of these different designs, at these two temperatures. FIG. 24d shows the comparison of peeling force as a function of swelling time for hydrogel adhesives with different designs: non-holey, holey, and holey with micro-channels for water at 35° C.

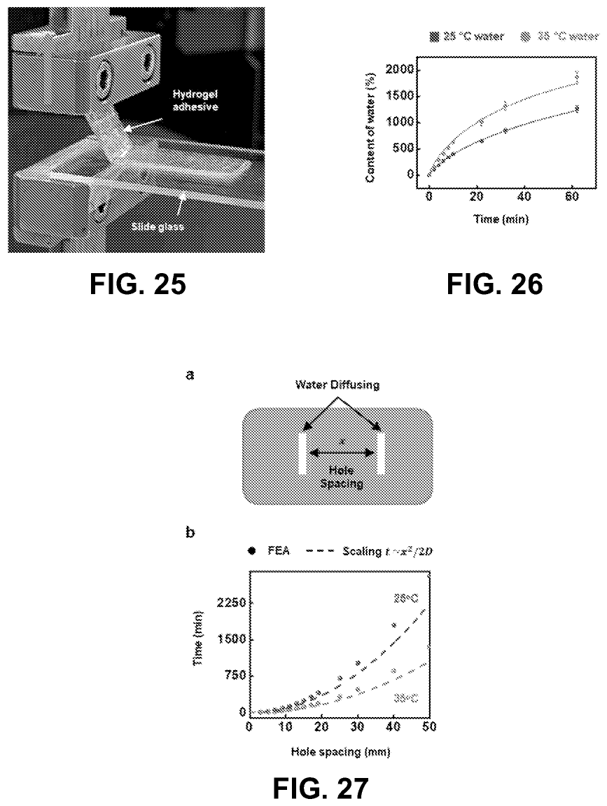

FIG. 24a shows the basic effect, measured as peel force required to remove a rectangular hydrogel adhesive (10×30 mm², thickness ˜815 μm) attached to a glass slide (FIG. 25), as a function of time of immersion in water at temperatures of 25° C. and 35° C. Specifically, FIG. 25 shows a photograph of the adhesion test setup for the hydrogel adhesive, using rectangular test structure (10×30 mm²) attached on a slide glass according to certain embodiments of the present invention. The diffusion coefficient for water through the hydrogel (˜1.2 mm²/min) at 35° C. is approximately twice as large as that (˜0.6 mm²/min) at 25° C., thereby resulting in a correspondingly higher swelling rate and a more rapid reduction in peel force (FIG. 26). Specifically, FIG. 26 shows the comparison of diffusion coefficients of a hydrogel adhesive (KM 40A) for water at 25° C. and 35° C. according to certain embodiments of the present invention. As shown in FIG. 26, the content of water corresponds to the change of the measured weight of the hydrogel adhesive over the swelling time for water at different temperatures. The diffusion coefficients are 5.56×10⁻⁷ m²/min for water at 25° C. and 1.18×10⁻⁶ m²/min for water at 35° C. Complete loss of adhesion, to within measurement uncertainties, occurs in 7 and 11 minutes at 35° C. and 25° C., respectively. The 35° C. case, close to the temperature of the skin, is most relevant for applications considered here.

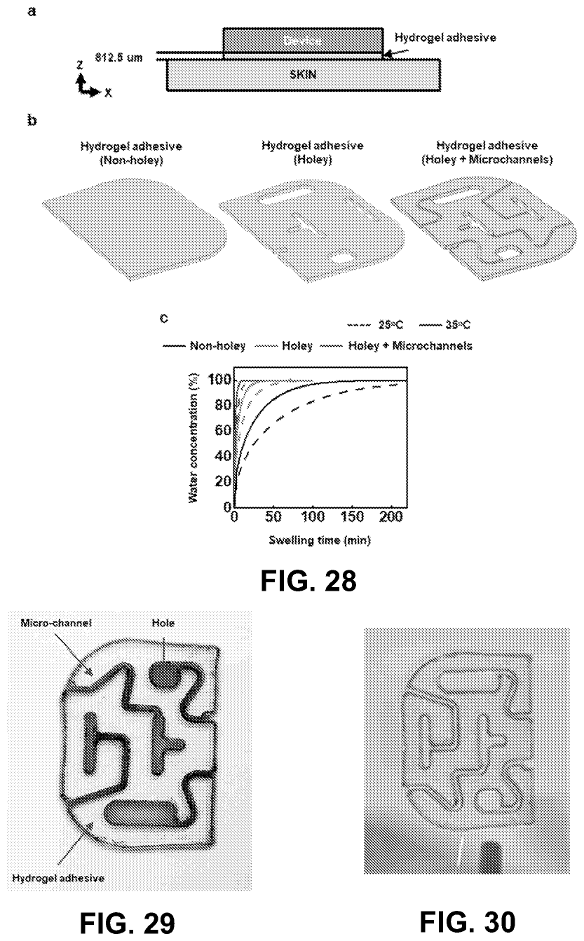

Without holes, this release mechanism is not practical because water can be introduced only around the perimeter of the device. The hydrogel adhesive consists of two separate pieces, one for each of the ECG electrodes. The widths of each piece are equal to half of that of the entire device. As such, the minimum diffusion distance is equal to one quarter of the width of the device (˜13.6 mm) for complete wetting of the hydrogel. The holes represent additional points of access that also simultaneously act as reservoirs, to reduce this distance. For the configuration of holes examined here, this reduction corresponds to a factor of ˜2 (˜6.7 mm). FIG. 27 shows the computed diffusion time as a function distance between the holes according to certain embodiments of the present invention. Specifically, FIG. 27a shows a schematic illustration of the hydrogel adhesive with two holes as a starting point for water diffusion. FIG. 27b shows the time when all the water reaches the hydrogel adhesive as a function of hole spacing, using measured diffusion coefficients (5.56×10⁻⁷ m²/min at 25° C. and 1.18×10⁻⁶ m²/min at 35° C.). Scaling between spacing and time follows the relationship t˜x²/2D t˜x²/2D where D is the diffusion coefficient, t is the diffusion time, and x is the distance between two holes, which is the diffusion distance. Given the quadratic dependence of diffusion distance on time in FIG. 27, this reduction translates to a decrease in the time to achieve saturation by a factor of ˜4. To reduce further the minimum diffusion distance and saturation time, the holes can serve as the origins for features that facilitate lateral transport (FIG. 28). Specifically, FIG. 28 shows the modeling results of diffusion for different hydrogel adhesive designs at 25° C. and 35° C. according to certain embodiments of the present invention. In particular, FIG. 28a shows a schematic illustration of a device attached on the skin using a hydrogel adhesive. FIG. 28b shows three hydrogel adhesive designs: non-holey, holey, and holey with microchannels. FIG. 28c shows the normalized water concentration as a function of time for hydrogel swelling at 25° C. and 35° C. for three hydrogel adhesive designs. Here, water introduced into the holes flows through microchannels (width: 500 μm, thickness: thickness 812.8 μm) cut into the hydrogel itself (FIGS. 29 and 30). Specifically, FIG. 29 schematically shows a photograph of a hydrogel adhesive design with holes and microchannels according to certain embodiments of the present invention. As shown in FIG. 29, the hydrogel adhesive with holes and microchannels is attached to the slide glass, and a transparent polymer film of holey design is covered on the hydrogel adhesive. Water with purple dye introduced into the holes flows through the microchannels. FIG. 30 shows water introduced into the holes and water passing through the microchannels in a hydrogel adhesive for a holey device with microchannel design according to certain embodiments of the present invention. These features further reduce the minimum diffusion distance (to ˜3 mm), corresponding to a reduction of saturation time by another factor of more than four (˜4.4 times) and by nearly twenty times compared to that of a conventional design, without holes or microchannels.

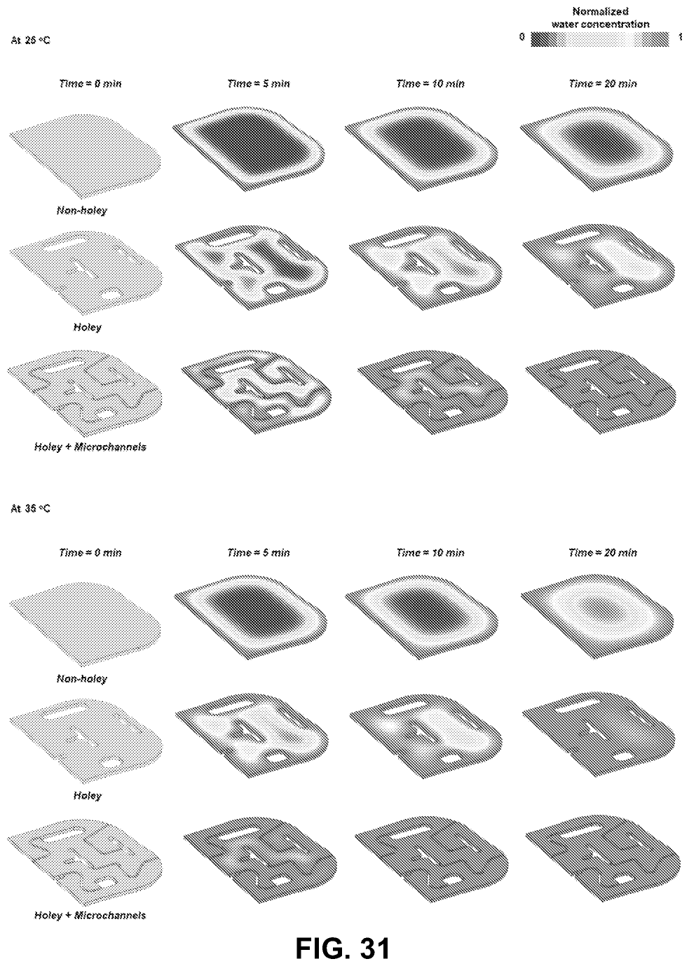

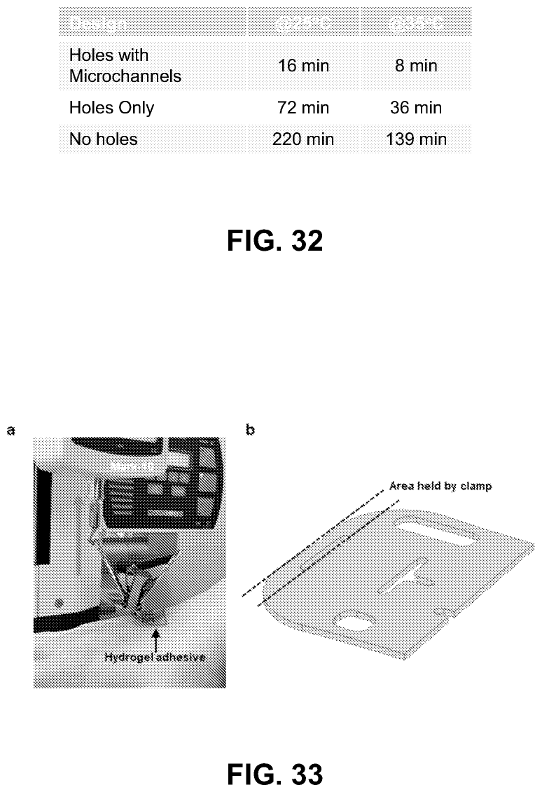

FIG. 24b-c and FIG. 31 show simulation results that use measured diffusion coefficients of hydrogel adhesive for water at 25° C. and 35° C. to compare these various cases (non-holey, holey, and holey with microchannels). Specifically, FIG. 31 shows the computed swelling model of water diffusion into the hydrogel adhesive as a function of time, water temperature, and adhesive design according to certain embodiments of the present invention, where (i) shows the holey design at 25° C., (ii) shows the holey design at 35° C. and (iii) shows the holey design with microchannels at 35° C. The hydrogel adhesive has an initial water content of 23%, as a baseline for calculating the normalized water concentration due to diffusion. Results of FEA capture the diffusion of water over the swelling time for each design of the hydrogel adhesive, as changes in the normalized water concentration with distance (line marked A in FIG. 24b) and through the entire volume (FIG. 24c). In the holey design, the center point of this line reaches a normalized water concentration of ˜20% and 50% at 10 min for temperatures of 25° C. and 35° C., respectively. For the holey design with microchannels, the concentration reaches 90% at 10 min for 35° C. FIG. 32 shows a table of the time to swell completely depending on hydrogel designs and water temperature according to certain embodiments of the present invention. Specifically, the table as shown in FIG. 32 shows the calculated saturation times for these three designs at 25 and 35° C. The holey design with microchannels at 35° C. exhibits a saturation time that is ˜27.5 times (8 min) smaller than that (220 min) of the non-holey case at 25° C.

Experimental measurements of peeling force for devices adhered to the back of the hand of an adult volunteer subject show trends consistent with modeling (FIG. 24d-e). Initially, all designs (non-holey, holey, and holey with microchannels) have peeling forces in the range of 5-7 N. Even after immersion in water at 35° C. for 10 minutes, the peeling force of the non-holey device shows the smallest reduction, still larger than 5.0 N, and the peeling force of the holey example is about 2.0 N. At that time, the peeling force of the holey with microchannels decreases by the largest amount, to a value less than 0.6 N. After 20 min, the device with holey design and microchannels exhibits zero adhesion, to within experimental uncertainties.

FIG. 33 shows the peeling test for a hydrogel adhesive on skin according to certain embodiments of the present invention. Specifically, FIG. 33a shows a photograph of a setup to measure the peeling force for a hydrogel adhesive attached to back of hand using a Mark-10instrument and clamp. FIG. 33b shows a schematic illustration of the area of the hydrogel adhesive held by clamp for peeling test.

### Moisture Release and Visual Inspection

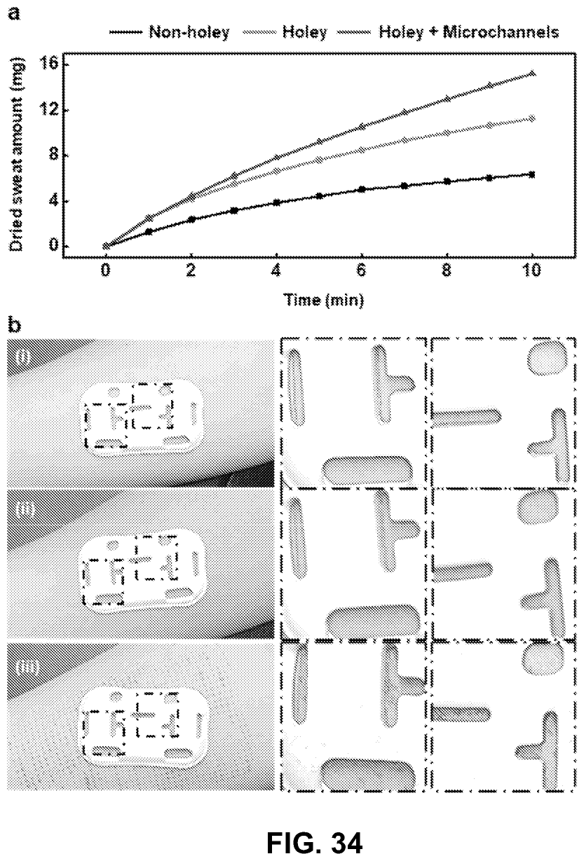

Just as the holes and microchannels facilitate permeation of water to the device-skin interface to trigger release of adhesion, permeation of moisture away from this interface via similar mechanisms can avoid the development of a moist environment in which fungi and bacteria can reproduce and cause allergic dermatitis. FIG. 34 schematically shows the measurements of breathability and demonstrations of visual inspection through the openings in a holey device according to certain embodiments of the present invention. Specifically, FIG. 34a shows the comparison of the weight of water lost by evaporation from a saturated hydrogel that lies between the base of a device and a glass slide substrate, as a function of time for devices with non-holey, holey, and holey with microchannels designs. FIG. 34b shows the images of devices with holey designs on (i) normal skin, (ii) skin reddened by irritation, (iii) skin with artificial red spots imitating allergic reaction for visual inspection of skin condition through holes.

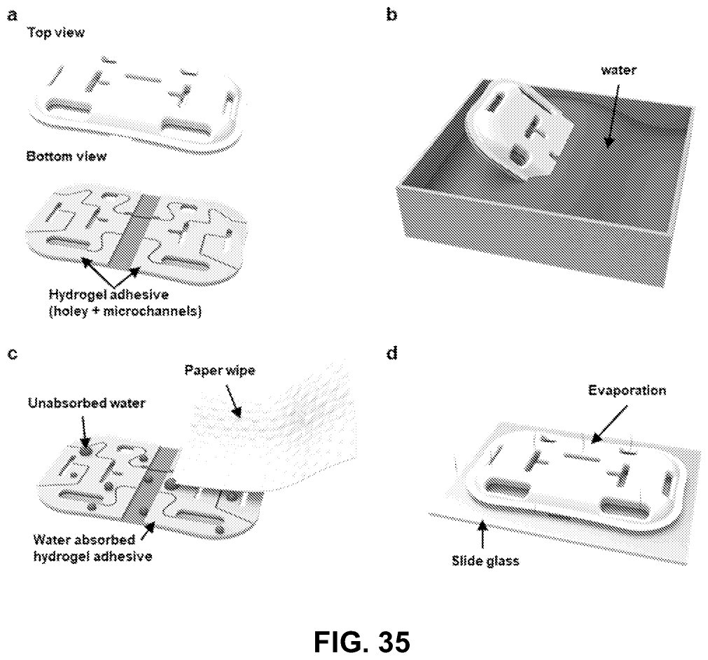

The results in FIG. 34a summarize measurements of that involve placing a device on top of a sample of fully hydrated hydrogel bonded to a glass slide and measuring the amount of weight of water lost by evaporation as a function of time (FIG. 35). Specifically, FIG. 35 schematically shows the testing protocol for evaluating water evaporation from the hydrogel adhesive according to certain embodiments of the present invention. In particular, FIG. 35a shows top and bottom view of a device with a pair of hydrogel adhesives. FIG. 35b shows the device with hydrogel adhesive is immersed in water and removed immediately. FIG. 35c shows the unabsorbed water is removed from the surface with a paper wipe. FIG. 35d shows the device with hydrogel adhesive is attached to a slide glass, and the amount of water evaporation is measured by weighing the structure at different time points. For the non-holey device, evaporation can only occur at the perimeter. For the holey device, evaporation occurs not only at the perimeter but also at the regions of the holes. The holey device with microchannels provides additional paths for water vapor to escape from the hydrogel. The findings suggest that the holey device offers breathability, as measured in this way, that is nearly two times (1.87) higher than the non-holey case. The enhancement for the holey device with microchannels corresponds to a factor of ˜2.4.

The holes also provide visual access for inspecting the condition of the skin, without removing the device. FIG. 34b show pictures of holey devices on (i) normal skin, (ii) skin reddened by irritation, and (iii) skin with artificial red spots to mimic allergic reactions. The changes in skin color are immediately apparent through the holes. This feature enables early diagnosis of the occurrence of rash or dermatitis of the skin interface.

### Operation in Capturing Electrocardiograms and High Bandwidth Accelerometry Data

The device described here supports single lead measurements of ECGs, tri-axis accelerations, and skin temperature. Data from the accelerometer yields SCGs, cycles of respiration (for determinations of RR), along with unique biomarkers derived from vibratory motions associated with crying and with coughing and wheezing, all without little effect of ambient sounds.

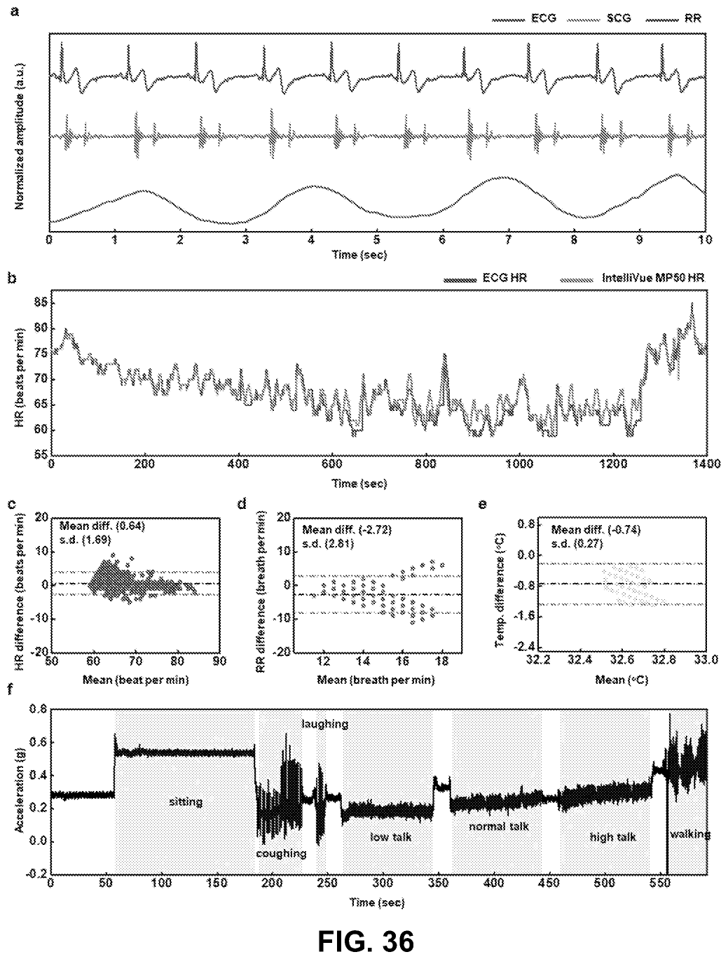

FIG. 36 shows representative data collected from a holey device according to certain embodiments of the present invention. Specifically, FIG. 36a shows representative ECGs, SCGs, and results of respiration (RR) wave forms captured from adult volunteer subjects with a holey device attached to their chests. The raw data continuously passes wirelessly to a mobile tablet that supports data analysis through a signal processing algorithm, for real-time graphical display. FIG. 36b shows the comparison of HR, RR and temperature to standard clinical measurements. Specifically, FIG. 36b shows 20 minutes of HR data compared to that collected with a clinical standard monitor (Intellivue MP50, Philips) in the intensive care unit. FIG. 36c-e shows corresponding Bland-Altman plots for (d) HR, (e) RR and (f) temperature, which present quantitative comparisons of HR, RR and temperature for three adult volunteer subjects using the Bland-Altman method. The mean difference and standard deviation (s.d.) are 0.64 and 1.69 beats per minute, −2.72 and 2.81 breaths per minute, and −0.74 and 0.27° C., respectively. The results for HR and RR are within +5 beats per minute and +3 breaths per minute, both compatible with regulatory guidelines set by the US FDA. The accelerometer also provides clinically important information on body motions/movements and orientation, along with vocal biomarkers and body sounds mentioned previously. FIG. 36f shows the data captured from the accelerometer from adult volunteer subject for various scenarios, including sitting, coughing, laughing, soft talking, normal talking, loud talking, and walking.

### Clinical Studies on a Neonatal Patient in the NICU

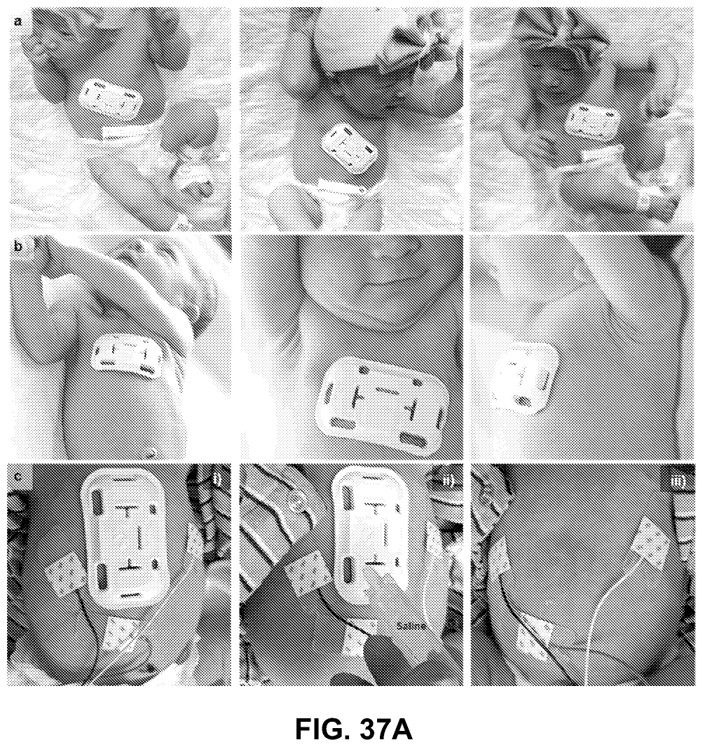

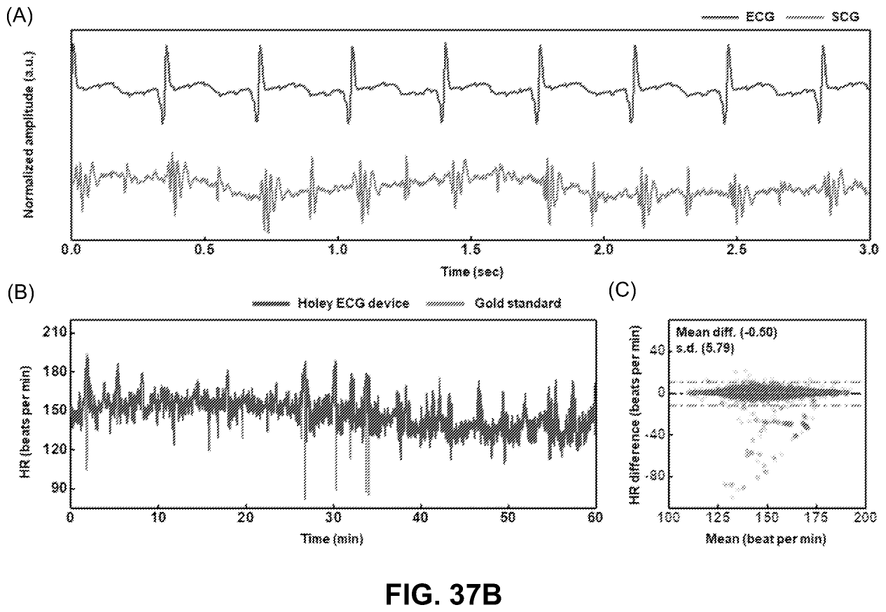

FIGS. 37A and 37B show clinical demonstrations of a holey device with the newborns and a neonatal patient in an operating NICU according to certain embodiments of the present invention. Specifically, FIG. 37A shows (a) representative photographs of the planar holey device on the newborns, (b) representative photographs of the 60° pre-curved holey device on the newborns, and (c) representative photographs of the holey device on a neonatal patient, who is a preterm infant (male, weight 2.56 kg, height 47 cm) of 38 weeks post menstrual age (36 weeks gestational age, 2 weeks chronological age) with diagnoses of prematurity and respiratory distress; i) when monitoring vital signs after mounting the device on the chest, ii) when applying saline through the holes to facilitate release of the hydrogel adhesive, iii) after the water-triggered soft release of the adhesive. The neonatal patient (male, weight 2.56 kg, height 47 cm) is 38 weeks post menstrual ages (36 weeks gestational age +2 weeks chronological age) with diagnoses of prematurity and respiratory distress of newborn. In FIG. 37A(a), the left photograph shows the holey ECG device attached to a chest of this patient using the hydrogel adhesive, the middle photograph shows the process of introducing the saline through holes for the water-triggered soft release of hydrogel adhesive after monitoring for 1 hour, and the right photograph shows the skin condition after removal of the device and the hydrogel adhesive. Although the part where the device is attached is slightly depressed, the device and the adhesive can be gently and easily removed without damaging the fragile skin. FIG. 37B shows (A) representative ECGs, SCGs captured the device attached to the chest, (B) comparison of HR determined with the device and with a gold standard system, and (C) corresponding Bland-Altman plot for HR. Specifically, FIG. 37B presents quantitative comparisons of HR for the neonatal patient using the Bland-Altman method. The mean difference and s.d. are −0.5 and 5.79, respectively, showing compatibility with guidelines set by the US FDA.

In conclusion, certain aspects of the present invention relate to thin, soft wireless devices that can continuously monitor key vital signs, such as HR, RR and temperature, and body motion/movements, with a focus on patients in the NICU and PICU. Key features include perforated (or holey) architectures, pre-curved layouts and structured hydrogel adhesives. The locations, dimensions and shapes of the holes improve the bending and stretching mechanics, to reduce irritation at the skin interface during device application, use and removal. Pre-curved shapes that match those of the targeted region of the anatomy further decrease the stresses on the surface of the skin, to values close to zero. In addition to these mechanical and geometrical advantages, the holey designs enable i) soft release of devices from the skin triggered by introduction of warm water through the holes and into microchannels molded on thin, hydrogel skin adhesives, ii) reduced accumulation of interfacial moisture from transepidermal water loss via enhanced rates of permeation through the holes, iii) visual inspection of the skin condition in the regions of the holes, without the need to remove the devices. Studies with adult volunteer subjects demonstrate the ability to capture HR, RR, and skin temperature with high levels of reliability and accuracy, along with measurements of body motions/movements. A clinical demonstration with a neonatal patient in an operating NICU shows that HR data from a holey device compare quantitatively to those collected using clinical standard apparatus and that the water-triggered soft release mechanism can be exploited in a commercial grade isolette, without significant change to routine procedures used with such patients. The outcomes of this work have the potential to enhance the quality of care for the neonates in all contexts, from the most advanced hospital settings to rural or remote health clinics in resource-constrained areas.

### Experimental Section/Methods

Fabrication and encapsulation of the holey device: A ISO 9001-compliant PCB manufacturer provided the fPCB for the holey device based on designs formed using Eagle CAD version 9 (Autodesk). Assembling components and programming the chips completed the functional electronics. The device includes passive components (resistors, inductors, and capacitors; footprint in inch from 0201 to 0603), power-management units (BQ25120A, Texas Instrument; TPS7A25, Texas Instrument), a BLE SoC (ISP1807-LR, Insight SIP), an ECG sensing unit (MAX30001, Maxim Integrated), an inertial measurement unit (LSM6DSL, STMicroelectronics) and a thermometer unit (MAX30205, Maxim Integrated). A folding process reduced the overall size of the device. A thin polyorganosiloxane elastomer film (Silbione RTV 4420, Elkem, mixed with 5 wt % blue silicone dye (Silc-Pig, Smooth-On)) formed by spin-casting at 250 rpm on a glass slide and thermal curing at 100° C. for 20 min. served as a bottom encapsulation layer. A cutting process with a CO₂ laser (ULS) defined two openings for the ECG electrodes and eight openings with different shapes for the holey design on the bottom layer. A silicone-based adhesive (3M 96042) attached the folded electronics to the bottom layer. A pair of convex (with holes) and concave (with pillars) aluminum molds for the top encapsulation layer of holey device was designed using SOLIDWORKS 2019 (Dassault Systemes), and printed using a milling machine (MDX-540, Rolland DGA). A thin (˜300 μm) polyorganosiloxane elastomer film (Silbione RTV 4420, mixed with 5 wt % white silicone dye) formed using these molds and thermal curing at 100° C. for 20 min defined the top layer. The top layer and the bottom layer with the electronics were overlaid, and the enclosed device was filled with an uncured polyorganosiloxane elastomer (Ecoflex 00-30, Smooth-On) to improve the softness and prevent tearing of the top and bottom layer by the fPCB, component chips and battery during mechanical deformations, such as bending and twisting. The completely enclosed device was thermally cured at 80° C. for 20 min and the edges of encapsulation layer were cut to complete the encapsulation process.

Mechanical simulation of the device: ABAQUS, a commercial finite element analysis (FEA) software, was used to model the mechanical behavior of the serpentine interconnects used for the electronics and the different encapsulation designs when subjected to different types of deformation (stretching, bending, and twisting). The objectives of the analysis were to 1) assess possibilities for plastic deformation (i.e., ϵ<0.3%) occurs in the copper serpentine interconnects when the device undergoes different types of external loads, 2) guide designs to reduce the magnitude of the strain in the optimized holey encapsulation, and 3) determine whether the interfacial normal and shear stresses imposed by the device onto the skin during deformation remain below the low somatosensory perception of the device on the skin for wearability, and 4) compare the resilience of the flat and pre-curved soft encapsulation designs associated with mounting on the curved surface of the skin. The thin copper (˜12 μm thick) and PI (˜25 μm thick) layers were modeled by composite shell elements (S4R), the soft encapsulation designs (with and without holes) were modeled by tetrahedron elements (C3D10), and the skin was modeled by hexahedron elements (C3D8R). The element size was tested to ensure convergence and accuracy of the simulation results. The elastic modulus (E) and Poisson's ratio (ν) were EPI =2.5 GPa and ν_{PI}=0.34 for PI; E_{Cu}=119 GPa and ν_{Cu}=0.34 for copper; E_{Silbione}=0.8 MPa and ν_{Silbione}=0.5 for Silbione; E_{Ecoflex}=60 kPa and ν_{Ecoflex}=0.5 for Ecoflex; and E_{Skin}=130 kPa and ν_{Skin}=0.5 for the skin.

Peeling force measurements of hydrogel adhesive on slide glass: Using the CO₂ laser (ULS), the hydrogel adhesive (KM 40A, Katecho) was cut into strips of 10 mm×30 mm, and then adhered to the slide glass. A solid analyzer (RSA G2, Tainstruments) determined the peeling force associated with detachment of the hydrogel adhesive from the surface of a slide glass. The upper mount of the analyzer held one edge of hydrogel adhesive strip, and the slide glass was fixed to the lower mount. The upper mount moved at 0.3 mm/s with the lower mount fixed, as the peeling force was recorded over time until the hydrogel adhesive strip was completely removed from the slide glass.

Diffusion coefficient of hydrogel adhesive: The hydrogel adhesive strip (10 mm×30 mm) was immersed in water at 25° C. and 35° C., and the change in weight of the hydrogel adhesive strip was recorded over time. The diffusion coefficient was determined to be 5.56×10⁻⁷ m²/min for water at 25° C. and 1.18×10⁻⁶ m²/min for water at 35° C. from non-linear fitting of the following equation:

$${C}_{t} / {C}_{∞} = 1 - exp [ - 7.3 {( D * t / {Z}^{2} )}^{0.75} ]$$

to experimental data, where Cₜ, C_{∞}, D, t, Z are the water content in a hydrogel adhesive strip at immersing time in water, the maximum water content, diffusion coefficient of water, immersing time in water, and thickness of the hydrogel adhesive strip, respectively.

Water diffusion simulation in the hydrogel: COMSOL, a commercial Multiphysics FEA software, was used to model the water concentration as a function of time in the non-holey, holey, and holey+microchannels hydrogel adhesive layouts at 25° C. and 35° C. The objective of the analysis was to determine the total diffusion time and to guide optimization of the holey+microchannel configuration for optimized rates of diffusion. The time-dependent diffusion was modeled according to

$$\frac{\partial c}{\partial t} + ∇ \cdot ( - D ∇ c ) = 0$$

where, D is the diffusion coefficient (m²/min), c is the water concentration (mol/m³), and t is time (min). The hydrogel (˜812.5 μm thick) was modeled using tetrahedral elements and the total number of elements in the model is ˜340,000. The diffusion model used in the simulation are determined from experiments as D=5.56×10⁻⁷ m²/min at 25° C. and D=1.18×10⁻⁶ m²/min at 35° C. Peeling force measurements of hydrogel adhesive on skin: Three designs (non-holey, holey, and holey with microchannels) of the hydrogel adhesive (KM 40A) were adhered to the pre-cleaned back of hand. A clamp held one edge of hydrogel adhesive, attached to a force gauge (Mark-10) with a motorized test stand (Mark-10) to measure the peeling force. The linear motor moved at 0.5 mm/s. The peeling force was recorded over time until the hydrogel adhesive was completely detached from the skin.

Water evaporation measurements of hydrogel adhesive: Three designs (non-holey, holey, and holey with microchannels) of the hydrogel adhesive (KM 40A) were adhered to the fully encapsulated holey device, and then immersed into water to allow the hydrogel adhesive to absorb the water. The unabsorbed water on the surface of hydrogel adhesive was removed with a paper wipe, and the device with the hydrogel adhesive was attached to a slide glass. At room temperature, water absorbed by the hydrogel adhesive evaporated and the resulting loss of weight was recorded over time.

Clinical study design: The feasibility of the wireless, holey chest sensor in a clinical setting was tested in a Level III NICU facility, under approved research protocols by the Institutional Review Boards of the Ann & Robert H. Lurie Children's Hospital of Chicago (IRB 2018-1668) and Northwestern University (STU00208150). After informed consent was obtained and proper sterilization of the device, according to approved procedures by the infection control committee of the Ann & Robert H. Lurie Children's Hospital of Chicago [Chung et al., Nat. Med., 26 (2020)], the sensor was gently placed onto the chest of the patient and adhered to the skin with two pieces of hydrogel adhesive (KM40A, Katecho). An iPad Pro with custom software applications, situated by the patient's bedside, was wirelessly connected to the sensor, allowing for continuous data collection and storage. A software application (MediCollector BEDSIDE) system was utilized to extract ECG data from the existing standard-of-care patient bedside monitor (Intellivue MP70, Philips). A drop of saline solution (0.9 wt % NaCl, Modudose) was added through each hole of the sensor to enable swelling of the underlying hydrogel adhesive and to facilitate release of the device from the skin.

In the embodiments as described above, the apparatus and the method utilizes the sensor systems with a pre-curved perforation architecture. However, the body parts of a mammal subject are mostly non-flat, and different areas of the body of the mammal subject may have different sizes and shapes. For example, on a human subject, the chest or forehead area may be relatively flatter, while the fingers may be more curved to require the apparatus to wrapp around an appendage. In another aspect of the invention, an apparatus for measuring physiological parameters of a mammal subject may utilize sensors that are by themselves bendable with memorable features, such that the sensors may be pre-curved to potentially provide better fit between the body locations of the mammal subject and the apparatus, in order to improve the sensibility of the sensors and the attaching stability as well as the measurement accuracy of the apparatus. Other features of such apparatus may include, without being limited thereto, better skin coupling, reduced lamination, less skin contact stress around the curvilinear surfaces of the mammal subject, and increased adjustability, adaptability and elasticity of the pre-curved architecture.

In certain embodiments, compared to the sensor systems in the embodiments as described above, the sensors being used in the apparatus may be formed (or at least partially formed) by a bendable shape-memory alloy (SMA) such that each sensor may have a pre-curved architecture with a non-zero curvature. The SMA is an alloy that can be deformed and then returns to its pre-deformed (or “remembered”) shape under certain conditions. For example, the SMA may be deformed at a cold temperature, and then returns to its pre-deformed shape when heated to a transformation temperature. In certain embodiments, the SMA may be a nickel-based SMA, an iron-based SMA, a copper-based SMA, or a combination thereof. In one embodiment, the shape-memory alloy may be nickel titanium, which is also known as nitonol. Nitonol exhibits the shape memory effect and superelasticity at different temperatures. Although other types of SMAs, such as iron-based and copper-based SMAs (e.g., Fe—Mn—Si, Cu—Zn—Al and Cu—Al—Ni), are commercially available and cheaper than nitonol, the nitonol (or NiTi-based SMAs) are widely used for most applications due to their stability and practicability and superior thermos-mechanic performance.

In certain embodiments, the apparatus for measuring physiological parameters of a mammal subject may include: a plurality of sensors being time-synchronized and communicate with each other wirelessly and bidirectionally, and a plurality of adhesive layers correspondingly disposed between the sensors and a skin of the mammal subject. Specifically, each sensor is bendable such that each sensor is configured to have a pre-curved architecture with a non-zero curvature, and each sensors is configured to detect a vital sign of the mammal subject and generate a corresponding one of the physiological parameters. Each adhesive layer is switchable chemically or physically between an adhesive state and a non-adhesive state, such that, for a corresponding sensor and a corresponding adhesive layer, the corresponding adhesive layer is configured to attach the corresponding sensor to a corresponding location on the mammal subject in the adhesive state, and to allow removal of the corresponding sensor system from the skin of the mammal subject when being switched to the non-adhesive state. Specifically, the non-zero curvature of the corresponding sensor is adjustable based on a shape of the corresponding location on the mammal subject. In certain embodiments, each sensor is at least partially formed by a bendable SMA.

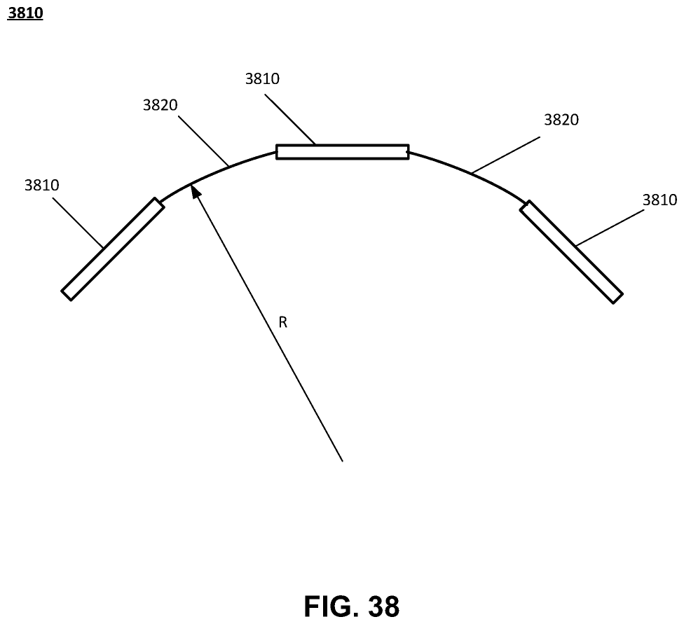

FIG. 38 schematically shows a sensor with a pre-curved architecture according to certain embodiments of the invention. Specifically, as shown in FIG. 38, the sensor 3800 is formed by three island regions 3810 and two flexible and stretchable interconnects 3820. In particular, each flexible and stretchable interconnects 3820 is interconnected between two adjacent island regions 3810, and each flexible and stretchable interconnects 3820 is formed by the SMA. The island regions 3810 are regions on which the electronic components of the sensors are disposed. For example, each sensor 3800 may be an EES, where the electronic components are disposed on each island regions 3810, and the flexible and stretchable interconnects 3820 are electrically connected to the electronic components on each island regions 3810. Since there are two interconnects 3820 provided, the overall architecture of the sensor 3800 can be pre-curved with a non-zero curvature. As shown in FIG. 38, the sensor 3800 has a radius R of curvature. Since the curvature of the sensor 3800 is not zero, the radius R of curvature is not infinity. The curvatures of the two interconnects 3820 may be preset by molding strategies to achieve a pre-curved architecture specifically fit to the corresponding location of the mammal subject on which the apparatus is to be disposed. Since the interconnects 3820 are formed by the SMA, the memorable feature of the SMA allows the interconnects 3820 to be flexible and stretchable while maintaining the possibility to retain the pre-curved architecture, and to have a memorable overall pre-curved architecture of the whole sensor 3800, thereby allowing the apparatus formed by multiple sensors 3800 to better fit on the skin of the mammal subject without being easily detached from the skin of the mammal subject.

In the embodiment as shown in FIG. 38, the sensor 3800 is formed by three island regions 3810 and two flexible and stretchable interconnects 3820 one dimensionally. In certain embodiments, however, the sensor 3800 may include more than three island regions 3810 and more than two flexible and stretchable interconnects 3820, and may be formed multi-dimensionally, such that a more complex pre-curved architecture may be achieved.

In the embodiments as described above, the SMA is used as the material of at least a part of the sensor 3800 to achieve the pre-curved architecture of the whole sensor 3800. In certain embodiments, other features may be applied to replace the SMA such that the sensor may be bendable to achieve the pre-curved architecture of the whole sensor 3800. For example, a cable and/or serpentines can be used to achieve a pre-curved architecture. Furthermore, a pre-curved architecture can be achieved simply with the silicone mold encapsulation.

In certain embodiments, the sensor 3800 may include perforations, such that the perforations of the sensor 3800 enable direct access to the adhesive layer between the sensor and the skin of the mammal subject. In other embodiments, it is also possible that the sensor 3800 does not include perforations. Other features of the apparatus utilizing such sensor 3800 may similar to the embodiments as discussed above, and are thus not further elaborated herein.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### REFERENCE LIST

[1] D. S. Wheeler, H. R. Wong, Pediatric Critical Care Medicine: Basic Science And Clinical Evidence, Springer Science & Business Media, Berlin, Germany, 2007.

[2] J. Xu, S. L. Murphy, K. D. Kochanek, B. Bastian, E. Arias, Natl. Vital Stat. Rep. 2018, 67.

[3] O. Bonner, K. Beardsall, N. Crilly, J. Lasenby, BMJ Innov. 2017, 3, 12.

[4] P. H. T. Cartlidge, P. E. Fox, N. Rutter, Early Hum. Dev. 1990, 21, 1.

[5] C. Lund, Newborn Infant Nurs. Rev. 2014, 14, 160.

[6] A. C. Tottman, J. M. Alsweiler, F. H. Bloomfield, J. E. Harding, Arch. Dis. Child Retal Neonatal Ed. 2018, 103, F277.

[7] D.-H. Kim, N. Lu, R. Ma, Y.-S. Kim, R.-H. Kim, S. Wang, J. Wu, S. M. Won, H. Tao, A. Islam, K. J. Yu, T. Kim, R. Chowdhury, M. Ying, L. Xu, M. Li, H.-J. Chung, H. Keum, M. McCormick, P. Liu, Y.-W. Zhang, F. G. Omenetto, Y. Huang, T. Coleman, J. A. Rogers, Science 2011, 333, 838.

[8] W. Gao, S. Emaminejad, H. Y. Y. Nyein, S. Challa, K. Chen, A. Peck, H. M. Fahad, H. Ota, H. Shiraki, D. Kiriya, D.-H. Lien, G. A. Brooks, R. W. Davis, A. Javey, Nature 2016, 529, 509.

[9] J. A. Walsh, E. J. Topol, S. R. Steinhubl, Circulation 2014, 130, 573.

[10] K.-I. Jang, K. Li, H. U. Chung, S. Xu, H. N. Jung, Y. Yang, J. W. Kwak, H. H. Jung, J. Song, C. Yang, A. Wang, Z. Liu, J. Y. Lee, B. H. Kim, J.-H. Kim, J. Lee, Y. Yu, B. J. Kim, H. Jang, K. J. Yu, J. Kim, J. W. Lee, J.-W. Jeong, Y. M. Song, Y. Huang, Y. Zhang, J. A. Rogers, Nat. Commun. 2017, 8, 15894.

[11] S. Y. Heo, J. Kim, P. Gutruf, A. Banks, P. Wei, R. Pielak, G. Balooch, Y. Shi, H. Araki, D. Rollo, C. Gaede, M. Patel, J. W. Kwak, A. E. Peña-Alcántara, K.-T. Lee, Y. Yun, J. K. Robinson, S. Xu, J. A. Rogers, Sci. Transl. Med. 2018, 10, eaau 1643.

[12] Y. Park, K. Kwon, S. S. Kwak, D. S. Yang, J. W. Kwak, H. Luan, T. S. Chung, K. S. Chun, J. U. Kim, H. Jang, H. Ryu, H. Jeong, S. M. Won, Y. J. Kang, M. Zhang, D. Pontes, B. R. Kampmeier, S. H. Seo, J. Zhao, I. Jung, Y. Huang, S. Xu, J. A. Rogers, Sci. Adv. 2020, 6,eabe1655.

[13] A. Y. Rwei, W. Lu, C. Wu, K. Human, E. Suen, D. Franklin, M. Fabiani, G. Gratton, Z. Xie, Y. Deng, S. S. Kwak, L. Li, C. Gu, A. Liu, C. M. Rand, T. M. Stewart, Y. Huang, D. E. Weese-Mayer, J. A. Rogers, PNAS 2020, 117, 31674.

[14] J. L. Vogl, E. C. Dunne, C. Liu, A. Bradley, A. Rwei, E. K. Lonergan, B. S. Hopkins, S. S. Kwak, C. D. Simon, C. M. Rand, J. A. Rogers, D. E. Weese-Mayer, C. F. Garfield, Dev. Psychobiol. DOI: 10.1002/dev.22100.

[15] H. Jeong, J. Y. Lee, K. Lee, Y. J. Kang, J.-T. Kim, R. Avila, A. Tzavelis, J. Kim, H. Ryu, S. S. Kwak, J. U. Kim, A. Banks, H. Jang, J.-K. Chang, S. Li, C. K. Mummidisetty, Y. Park, S. Nappi, K. S. Chun, Y. J. Lee, K. Kwon, X. Ni, H. U. Chung, H. Luan, J.-H. Kim, C. Wu, S. Xu, A. Banks, A. Jayaraman, Y. Huang, J. A. Rogers, Sci. Adv. 2021, 7, eabg3092.

[16] C. Liu, J.-T. Kim, S. S. Kwak, A. Hourlier-Fargette, R. Avila, J. Vogl, A. Tzavelis, H. U. Chung, J. Y. Lee, D. H. Kim, D. Ryu, K. B. Fields, J. L. Ciatti, S. Li, M. Irie, A. Bradley, A. Shukla, J. Chavez, E. C. Dunne, S. S. Kim, J. Kim, J. B. Park, H. H. Jo, J. Kim, M. C. Johnson, J. W. Kwak, S. R. Madhvapathy, S. Xu, C. M. Rand, L. E. Marsillio, S. J. Hong, Y. Huang, D. E. Weese-Mayer, J. A. Rogers, Adv. Healthc. Mater. DOI: 10.1002/adhm.202100383.

[17] H. U. Chung, B. H. Kim, J. Y. Lee, J. Lee, Z. Xie, E. M. Ibler, K. Lee, A. Banks, J. Y. Jeong, J. Kim, C. Ogle, D. Grande, Y. Yu, H. Jang, P. Assem, D. Ryu, J. W. Kwak, M. Namkoong, J. B. Park, Y. Lee, D. H. Kim, A. Ryu, J. Jeong, K. You, B. Ji, Z. Liu, Q. Huo, X. Feng, Y. Deng, Y. Xu, K.-I. Jang, J. Kim, Y. Zhang, R. Ghaffari, C. M. Rand, M. Schau, A. Hamvas, D. E. Weese-Mayer, Y. Huang, S. M. Lee, C. H. Lee, N. R. Shanbhag, A. S. Paller, S. Xu, J. A. Rogers, Science 2019, 363, eaau0780.

[18] H. U. Chung, A. Y. Rwei, A. Hourlier-Fargette, S. Xu, K. Lee, E. C. Dunne, Z. Xie, C. Liu, A. Carlini, D. H. Kim, D. Ryu, E. Kulikova, J. Cao, I. C. Odland, K. B. Fields, B. Hopkins, A. Banks, C. Ogle, D. Grande, J. B. Park, J. Kim, M. Irie, H. Jang, J. Lee, Y. Park, J. Kim, H. H. Jo, H. Hahm, R. Avila, Y. Xu, M. Namkoong, J. W. Kwak, E. Suen, M. A. Paulus, R. J. Kim, B. V. Parsons, K. A. Human, S. S. Kim, M. Patel, W. Reuther, H. S. Kim, S. H. Lee, J. D. Leedle, Y. Yun, S. Rigali, T. Son, I. Jung, H. Arafa, V. R. Soundararajan, A. Ollech, A. Shukla, A. Bradley, M. Schau, C. M. Rand, L. E. Marsillio, Z. L. Harris, Y. Huang, A. Hamvas, A. S. Paller, D. E. Weese-Mayer, J. Y. Lee, J. A. Rogers, Nat. Med. 2020, 26, 418.

[19] M. C. Mahoney, M. I. Cohen, Pediat. Phys. Ther. 2005, 17, 194.

[20] Y. Shinya, M. Kawai, F. Niwa, M. Imafuku, M. Myowa, Front. Psychol 2017, 8, 2195.

[21] M. Y. Hadush, A. H. Berhe, A. A. Medhanyie, BMC Pediatr. 2017, 17, 111.

[22] C. W. Peak, J. J. Wilker, G. Schmidt, Colloid Polym. Sci. 2013, 291, 2031.

[23] R. Michel, L. Poirier, Q. van Poelvoorde, J. Legagneux, M. Manassero, L. Corté, PNAS 2019, 116, 738.

[24] X. Du, Y. Hou, L. Wu, S. Li, A. Yu, D. Kong, L. Wang, G. Niu, J. Mater. Chem. B 2020, 8, 5682.

[25] A. Miyamoto, S. Lee, N. F. Cooray, S. Lee, M. Mori, N. Matsuhisa, H. Jin, L. Yoda, T. Yokota, A. Itoh, M. Sekino, H. Kawasaki, T. Ebihara, M. Amagai, T. Someya, Nat. Nanotechnol. 2017, 12, 907.

[26] S. Yoon, M. Seok, M. Kim, Y.-H. Cho, Sci. Rep. 2021, 11, 938.

[27] K. Lee, X. Ni, J. Y. Lee, H. Arafa, D. J. Pe, S. Xu, R. Avila, M. Irie, J. H. Lee, R. L. Easterlin, D. H. Kim, H. U. Chung, O. O. Olabisi, S. Getaneh, E. Chung, M. Hill, J. Bell, H. Jang, C. Liu, J. B. Park, J. Kim, S. B. Kim, S. Mehta, M. Pharr, A. Tzavelis, J. T. Reeder, I. Huang, Y. Deng, Z. Xie, C. R. Davies, Y. Huang, J. A. Rogers, Nat. Biomed. Eng. 2020, 4, 148.

[28] A. Kaneko, N. Asai, T. Kanda, J. Hand Ther. 2005, 18, 421.

[29] S. Wang, M. Li, J. Wu, D.-H. Kim, N. Lu, Y. Su, Z. Kang, Y. Huang, J. A. Rogers, J. Appl. Mech. 2012, 79, 031022.

[30] C. H. Lee, Y. Ma, K.-I. Jang, A. Banks, T. Pan, X. Feng, J. S. Kim, D. Kang, M. S. Raj, B. L. McGrane, B. Morey, X. Wang, R. Ghaffari, Y. Huang, J. A. Rogers, Adv. Funct. Mater. 2015, 25, 3698.

[31] A. Koh, D. Kang, Y. Xue, S. Lee, R. M. Pielak, J. Kim, T. Hwang, S. Min, A. Banks, P. Bastien, M. C. Manco, L. Wang, K. R. Ammann, K.-I. Jang, P. Won, S. Han, R. Ghaffari, U. Paik, M. J. Slepian, G. Balooch, Y. Huang, J. A. Rogers, Sci. Transl. Med. 2016, 8, 366ra165.

[32] D. L. August, K. New, R. A. Ray, Y. Kandasamy, J. Neonatal Nurs. 2018, 24, 173.

[33] T. Oranges, V. Dini, M. Romanelli, Adv. Wound Care 2015, 4, 587.

[34] S. Patil, S. Patil, P. Durgawale, J. Datta. Meghe Inst. Med. Sci. Univ. 2018, 13, 143.

[35] I. G. Azevedo, N. S. O. Holanda, N. M. R. Arrais, R. T. G. Santos, A. G. F. Araujo, S. A. Pereira, BMC Pediatr. 2019, 19, 341.

[36] Y. Zhang, R. C. Webb, H. Luo, Y. Xue, J. Kurniawan, N. H. Cho, S. Krishnan, Y. Li, Y. Huang, J. A. Rogers, Adv. Healthc. Mater. 2016, 5, 119.

[37] Y. M. Lee, S. H. Kim, C. S. Cho, J. Appl. Polym. Sci. 1996, 62, 301.

[38] S. J. Kim, S. J. Park, S. I. Kim, React. Funct. Polym. 2003, 55, 53.

[39] J. Crank, The Mathematics of Diffusion, Second Edition, Oxford University Press, NY, USA, 1975.

[40] C.-H. Shen, G. S. Springer, J. Compos. Mater. 1976, 10, 2.

[41] H. S. Jang, G.-G. Kim, S. H. Kang, Y. Kim, J. L. Yoo, S. Yoo, K.-K. Kim, C. Jung, H. C. Ko, Adv. Mater. 2018, 30, 1801256.

[42] J. Martin Bland, DouglasG. Altman, Lancet 1986, 327, 307.

What is claimed is:

1. An apparatus for measuring physiological parameters of a mammal subject, comprising:
a plurality of sensors configured to be time-synchronized and in communication with each other wirelessly and bidirectionally, wherein each of the sensors is bendable such that each of the sensors is configured to have a pre-curved architecture with a non-zero curvature, and each of the sensors is configured to detect a vital sign of the mammal subject and generate a corresponding one of the physiological parameters; and
a plurality of adhesive layers, configured to be disposed between the sensors and a skin of the mammal subject correspondingly, wherein each of the adhesive layers is switchable chemically or physically between an adhesive state and a non-adhesive state, such that, for a corresponding sensor of the sensors and a corresponding adhesive layer of the adhesive layers, the corresponding adhesive layer is configured to attach the corresponding sensor to a corresponding location on the mammal subject in the adhesive state, and to allow removal of the corresponding sensor system from the skin of the mammal subject when being switched to the non-adhesive state;
wherein the non-zero curvature of the corresponding sensor is configured to be adjustable based on a shape of the corresponding location on the mammal subject.

2. The apparatus of claim 1, wherein each of the sensors is at least partially formed by a bendable shape-memory alloy (SMA).

3. The apparatus of claim 2, wherein each of the sensors is formed by at least three island regions and at least two flexible and stretchable interconnects, each of the at least two flexible and stretchable interconnects is interconnected between two adjacent island regions of the at least three island regions, and each of the at least two flexible and stretchable interconnects is formed by the SMA.

4. The apparatus of claim 3, wherein each of the sensors is an epidermal electronic system (EES) comprising:
a middle circuit board layer including a plurality of electronic components disposed on each of the at least three island regions, wherein the at least two flexible and stretchable interconnects are electrically connected to different ones of the electronic components; and
a top elastomeric encapsulation layer and a bottom elastomeric encapsulation layer sandwiching the middle circuit board layer, wherein the bottom elastomeric encapsulation layer forms a tissue-facing surface attached to the mammal subject, and the top elastomeric encapsulation layer forms an environment-facing surface.

5. The apparatus of claim 2, wherein the SMA is a nickel-based SMA, an iron-based SMA, a copper-based SMA, or a combination thereof.

6. The apparatus of claim 5, wherein the SMA is nitonol.

7. The apparatus of claim 1, wherein each of the sensors includes a plurality of perforations, such that the perforations of the corresponding sensor are configured to enable direct access to the corresponding adhesive layer.

8. The apparatus of claim 7, wherein for each of the sensors, the perforations are located to allow direct visualization of the skin of the mammal subject underlying the sensors without removal of the sensors.

9. The apparatus of claim 7, wherein each of the adhesive layers is switchable chemically from the adhesive state to the non-adhesive state by applying a liquid or a chemical solution directly to the adhesive layers through the perforations.

10. The apparatus of claim 9, wherein the liquid or the chemical solution includes:
water,
normal saline,
a solution with a certain level of pH value,
a solution for dissolving the adhesive layers through thiol-disulfide exchange reactions, retro-Michael reactions, or retro Diels-Alder reactions, and
a glucose solution for dissolving the adhesive layers.

11. The apparatus of claim 1, wherein each of the adhesive layers is switchable physically from the adhesive state to the non-adhesive state through a thermal process or light.

12. The apparatus of claim 1, wherein each of the adhesive layers is formed by a hydrogel adhesive, hydrocolloid adhesives, a polymeric film, fiber adhesives, or an acrylic adhesive.

13. The apparatus of claim 1, wherein each of the sensors is configured to detect the vital sign as a signal including one of:
an electrical signal related to at least one of electrocardiography (ECG) and electromyography (EMG) technology;
a mechanical signal related to movement, respiration and arterial tonometry;
an acoustic signal related to vocal cord vocalization, respiratory sound and heart sound; and
an optical signal related to at least one of blood oxygenation and blood pressure.

14. The apparatus of claim 1, wherein each of the sensors further comprises a power supply, and the power supply is an embedded power supply or a detachable modular power supply.

15. The apparatus of claim 1, further comprising a microcontroller unit (MCU) configured to be in wireless communication with the plurality of sensors, and configured to receive the physiological parameters of the mammal subject from the sensors and to display the physiological parameters of the mammal subject.

16. The apparatus of claim 15, wherein each of the sensors is configured to be in wireless communication with the MCU via a near field communication (NFC) protocol, or Bluetooth protocol.

17. The apparatus of claim 16, wherein each of the sensors comprises a magnetic coil in compliance with the NFC protocol to allow wireless data transmission and wireless power transmission through a single link.

18. The apparatus of claim 1, wherein each of the plurality of sensors further comprises one or more of:
an accelerometer for position or movement monitoring; and
a temperature sensor for measuring temperature.

19. The apparatus of claim 1, wherein the physiological parameters of the mammal subject comprise one or more of: heart rate, heart rate variability, heart sounds, blood pressure, chest wall displacement, electromyography, electrocardiography, blood oxygenation, respiratory rate, respiratory effort, respiratory cadence, tidal volume, coughing, snoring, sneezing, throat clearing, wheezing, apnea, hypoapnea, physical activity, core body position, peripheral limb position, scratching, vocalizations, rubbing, walking, sleep quality, sleep time, wake time upon sleeping, skin temperature, core body temperature, and a combination thereof.

20. The apparatus of claim 1, wherein the mammal subject is a human subject or a non-human subject.

21. A method of measuring physiological parameters of a mammal subject, the method comprising:
attaching, by a plurality of adhesive layers in an adhesive state, a plurality of sensors on the mammal subject, wherein the adhesive layers are correspondingly disposed between the sensor systems and a skin of the mammal subject, each of the adhesive layers is switchable chemically or physically between the adhesive state and a non-adhesive state, the sensors are configured to be time-synchronized and in communication with each other wirelessly and bidirectionally, each of the sensors is bendable such that each of the sensors is configured to have a pre-curved architecture with a non-zero curvature, and each of the sensors is configured to monitor one of the physiological parameters;
measuring, by the sensors, the physiological parameters of the mammal subject; and
in response to a need to remove a corresponding sensor of the sensors from a corresponding location on the mammal subject, switching chemically or physically a corresponding adhesive layer of the adhesive layers to the non-adhesive state to remove the corresponding sensor from the skin of the mammal subject,
wherein the non-zero curvature of the corresponding sensor is adjustable based on a shape of the corresponding location on the mammal subject.

22. The method of claim 21, wherein each of the sensors is at least partially formed by a bendable shape-memory alloy (SMA).

23. The method of claim 22, wherein each of the sensors is formed by at least three island regions and at least two flexible and stretchable interconnects, each of the at least two flexible and stretchable interconnects is interconnected between two adjacent island regions of the at least three island regions, and each of the at least two flexible and stretchable interconnects is formed by the SMA.

24. The method of claim 23, wherein each of the sensors is an epidermal electronic system (EES) comprising:
a middle circuit board layer including a plurality of electronic components disposed on each of the at least three island regions, wherein the at least two flexible and stretchable interconnects are electrically connected to different ones of the electronic components; and
a top elastomeric encapsulation layer and a bottom elastomeric encapsulation layer sandwiching the middle circuit board layer, wherein the bottom elastomeric encapsulation layer forms a tissue-facing surface attached to the mammal subject, and the top elastomeric encapsulation layer forms an environment-facing surface.

25. The method of claim 22, wherein the SMA is a nickel-based SMA, an iron-based SMA, a copper-based SMA, or a combination thereof.

26. The method of claim 25, wherein the SMA is nitonol.

27. The method of claim 21, wherein each of the sensors includes a plurality of perforations, such that the perforations of the corresponding sensor system are configured to enable direct access to the corresponding adhesive layer.

28. The method of claim 27, wherein for each of the sensors, the perforations are located to allow direct visualization of the skin of the mammal subject underlying the sensor systems without removal of the sensor systems.

29. The method of claim 27, wherein each of the adhesive layers is switchable chemically from the adhesive state to the non-adhesive state by applying a liquid or a chemical solution directly to the adhesive layers through the perforations.

30. The method of claim 29, wherein the liquid or the chemical solution includes:
water,
normal saline,
a solution with a certain level of pH value,
a solution for dissolving the adhesive layers through thiol-disulfide exchange reactions, retro-Michael reactions, or retro Diels-Alder reactions, and
a glucose solution for dissolving the adhesive layers.

31. The method of claim 21, wherein the switching of the corresponding adhesive layer the non-adhesive state is performed by:
applying a thermal process or light to the corresponding adhesive layer.

32. The method of claim 21, wherein each of the adhesive layers is formed by a hydrogel adhesive, hydrocolloid adhesives, a polymeric film, fiber adhesives, or an acrylic adhesive.

33. The method of claim 21, wherein the sensor is configured to detect the vital sign as a signal including one of:
an electrical signal related to at least one of electrocardiography (ECG) and electromyography (EMG) technology;
a mechanical signal related to movement, respiration and arterial tonometry;
an acoustic signal related to vocal cord vocalization, respiratory sound and heart sound; and
an optical signal related to at least one of blood oxygenation and blood pressure.

34. The method of claim 21, wherein each of the sensors further comprises a power supply, and the power supply is an embedded power supply or a detachable modular power supply.

35. The method of claim 21, wherein the sensors are configured to be in wireless communication with a microcontroller unit (MCU).

36. The method of claim 35, further comprising:
receiving, at the MCU, the physiological parameters of the mammal subject; and
displaying, at the MCU, the physiological parameters of the mammal subject.

37. The method of claim 21, wherein the physiological parameters of the mammal subject comprise one or more of: heart rate, heart rate variability, heart sounds, blood pressure, chest wall displacement, electromyography, electrocardiography, blood oxygenation, respiratory rate, respiratory effort, respiratory cadence, tidal volume, coughing, snoring, sneezing, throat clearing, wheezing, apnea, hypoapnea, physical activity, core body position, peripheral limb position, scratching, vocalizations, rubbing, walking, sleep quality, sleep time, wake time upon sleeping, skin temperature, core body temperature, and a combination thereof.

38. A non-transitory tangible computer-readable medium storing instructions which, when executed by one or more processors, cause the method of claim 21 to be performed.

39. An apparatus for measuring physiological parameters of a mammal subject, comprising:
a plurality of sensor systems configured to be time-synchronized and in communication with each other wirelessly and bidirectionally, wherein each of the sensor systems comprises a flexible printed circuit board (fPCB) with a pre-curved perforated architecture and at least one sensor disposed on the fPCB, and the at least one sensor is configured to detect a vital sign of the mammal subject and generate a corresponding one of the physiological parameters; and
a plurality of adhesive layers, configured to be disposed between the sensor systems and a skin of the mammal subject correspondingly, wherein each of the adhesive layers has a perforated architecture and is switchable chemically or physically between an adhesive state and a non-adhesive state, such that, for a corresponding sensor system of the sensor systems and a corresponding adhesive layer of the adhesive layers, the corresponding adhesive layer is configured to attach the corresponding sensor system to the mammal subject in the adhesive state, and to allow removal of the corresponding sensor system from the skin of the mammal subject when being switched to the non-adhesive state.

40. The apparatus of claim 39, wherein each of the sensor systems includes a plurality of perforations, such that the perforations of the corresponding sensor system are configured to enable direct access to the corresponding adhesive layer.

41. The apparatus of claim 40, wherein for each of the sensor systems, the perforations are located to allow direct visualization of the skin of the mammal subject underlying the sensor systems without removal of the sensor systems.

42. The apparatus of claim 40, wherein each of the sensor systems is an epidermal electronic system (EES) comprising:
a middle circuit board layer including a plurality of electronic components, and a plurality of flexible and stretchable interconnects electrically connected to different electronic components; and
a top elastomeric encapsulation layer and a bottom elastomeric encapsulation layer sandwiching the middle circuit board layer, wherein the bottom elastomeric encapsulation layer forms a tissue-facing surface attached to the mammal subject, and the top elastomeric encapsulation layer forms an environment-facing surface.

43. The apparatus of claim 42, wherein the middle circuit board layer is formed by a foldable electronic board, and the plurality of electronic components and the plurality of flexible and stretchable interconnects are disposed on the foldable electronic board.

44. The apparatus of claim 42, wherein the perforations of each of the sensor systems are formed by corresponding perforations formed on each of the top elastomeric encapsulation layer, the middle circuit board layer and the elastomeric encapsulation layer, the corresponding perforations formed on the middle circuit board layer exists between the electronic components, and the corresponding perforations formed on the top elastomeric encapsulation layer and the bottom elastomeric encapsulation layer integrate and align with the corresponding perforations formed on the middle circuit board layer.

45. The apparatus of claim 40, wherein each of the adhesive layers is switchable chemically from the adhesive state to the non-adhesive state by applying a liquid or a chemical solution directly to the adhesive layers through the perforations.

46. The apparatus of claim 45, wherein the liquid or the chemical solution includes:
water,
normal saline,
a solution with a certain level of pH value,
a solution for dissolving the adhesive layers through thiol-disulfide exchange reactions, retro-Michael reactions, or retro Diels-Alder reactions, and
a glucose solution for dissolving the adhesive layers.

47. The apparatus of claim 39, wherein each of the adhesive layers is switchable physically from the adhesive state to the non-adhesive state through a thermal process or light.

48. The apparatus of claim 39, wherein each of the adhesive layers is formed by a hydrogel adhesive, hydrocolloid adhesives, a polymeric film, fiber adhesives, or an acrylic adhesive.

49. The apparatus of claim 39, wherein the sensor is configured to detect the vital sign as a signal including one of:
an electrical signal related to at least one of electrocardiography (ECG) and electromyography (EMG) technology;
a mechanical signal related to movement, respiration and arterial tonometry;
an acoustic signal related to vocal cord vocalization, respiratory sound and heart sound; and
an optical signal related to at least one of blood oxygenation and blood pressure.

50. The apparatus of claim 39, wherein each of the sensor systems further comprises a power supply, and the power supply is an embedded power supply or a detachable modular power supply.

51. The apparatus of claim 39, further comprising a microcontroller unit (MCU) configured to be in wireless communication with the plurality of sensor systems, and configured to receive, from the sensor systems, and to display the physiological parameters of the mammal subject.

52. The apparatus of claim 51, wherein each of the sensor systems is configured to be in wireless communication with the MCU via a near field communication (NFC) protocol, or Bluetooth protocol.

53. The apparatus of claim 52, wherein each of the sensor systems comprises a magnetic coil in compliance with the NFC protocol to allow wireless data transmission and wireless power transmission through a single link.

54. The apparatus of claim 39, wherein each of the plurality of sensor systems further comprises one or more of:
an accelerometer for position or movement monitoring; and
a temperature sensor for measuring temperature.

55. The apparatus of claim 39, wherein the physiological parameters of the mammal subject comprise one or more of: heart rate, heart rate variability, heart sounds, blood pressure, chest wall displacement, electromyography, electrocardiography, blood oxygenation, respiratory rate, respiratory effort, respiratory cadence, tidal volume, coughing, snoring, sneezing, throat clearing, wheezing, apnea, hypoapnea, physical activity, core body position, peripheral limb position, scratching, vocalizations, rubbing, walking, sleep quality, sleep time, wake time upon sleeping, skin temperature, core body temperature, and a combination thereof.

56. The apparatus of claim 39, wherein the mammal subject is a human subject or a non-human subject.

57. A method of measuring physiological parameters of a mammal subject, the method comprising:
attaching, by a plurality of adhesive layers in an adhesive state, a plurality of sensor systems on the mammal subject, wherein the adhesive layers are correspondingly disposed between the sensor systems and a skin of the mammal subject, each of the adhesive layers has a pre-curved perforated architecture and is switchable chemically or physically between the adhesive state and a non-adhesive state, the sensor systems are configured to be time-synchronized and in communication with each other wirelessly and bidirectionally, and each of the sensor systems comprises a flexible printed circuit board (fPCB) with a perforated architecture and at least one sensor disposed on the fPCB to monitor one of the physiological parameters;
measuring, by the sensor systems, the physiological parameters of the mammal subject; and
in response to a need to remove a corresponding sensor system of the sensor systems, switching chemically or physically a corresponding adhesive layer of the adhesive layers to the non-adhesive state to remove the corresponding sensor system from the skin of the mammal subject.

58. The method of claim 57, wherein each of the sensor systems includes a plurality of perforations, such that the perforations of the corresponding sensor system are configured to enable direct access to the corresponding adhesive layer.

59. The method of claim 58, wherein for each of the sensor systems, the perforations are located to allow direct visualization of the skin of the mammal subject underlying the sensor systems without removal of the sensor systems.

60. The method of claim 58, wherein each of the sensor systems is an epidermal electronic system (EES) comprising:
a middle circuit board layer including a plurality of electronic components, and a plurality of flexible and stretchable interconnects electrically connected to different electronic components; and
a top elastomeric encapsulation layer and a bottom elastomeric encapsulation layer sandwiching the middle circuit board layer, wherein the bottom elastomeric encapsulation layer forms a tissue-facing surface attached to the mammal subject, and the top elastomeric encapsulation layer forms an environment-facing surface.

61. The method of claim 60, wherein the middle circuit board layer is formed by a foldable electronic board, and the plurality of electronic components and the plurality of flexible and stretchable interconnects are disposed on the foldable electronic board.

62. The method of claim 60, wherein the perforations of each of the sensor systems are formed by corresponding perforations formed on each of the top elastomeric encapsulation layer, the middle circuit board layer and the elastomeric encapsulation layer, the corresponding perforations formed on the middle circuit board layer exists between the electronic components, and the corresponding perforations formed on the top elastomeric encapsulation layer and the bottom elastomeric encapsulation layer integrate and align with the corresponding perforations formed on the middle circuit board layer.

63. The method of claim 58, wherein the switching of the corresponding adhesive layer the non-adhesive state is performed by:
applying a liquid or a chemical solution directly to the corresponding adhesive layer through the perforations of the corresponding sensor system.

64. The method of claim 63, wherein the liquid or the chemical solution includes:
water,
normal saline,
a solution with a certain level of pH value,
a solution for dissolving the adhesive layers through thiol-disulfide exchange reactions, retro-Michael reactions, or retro Diels-Alder reactions, and
a glucose solution for dissolving the adhesive layers.

65. The method of claim 57, wherein the switching of the corresponding adhesive layer the non-adhesive state is performed by:
applying a thermal process or light to the corresponding adhesive layer.

66. The method of claim 57, wherein each of the adhesive layers is formed by a hydrogel adhesive, hydrocolloid adhesives, a polymeric film, fiber adhesives, or an acrylic adhesive.

67. The method of claim 57, wherein the sensor is configured to detect the vital sign as a signal including one of:
an electrical signal related to at least one of electrocardiography (ECG) and electromyography (EMG) technology;
a mechanical signal related to movement, respiration and arterial tonometry;
an acoustic signal related to vocal cord vocalization, respiratory sound and heart sound; and
an optical signal related to at least one of blood oxygenation and blood pressure.

68. The method of claim 57, wherein each of the sensor systems further comprises a power supply, and the power supply is an embedded power supply or a detachable modular power supply.

69. The method of claim 57, wherein the sensor systems are configured to be in wireless communication with a microcontroller unit (MCU).

70. The method of claim 69, further comprising:
receiving, at the MCU, the physiological parameters of the mammal subject; and
displaying, at the MCU, the physiological parameters of the mammal subject.

71. The method of claim 57, wherein the physiological parameters of the mammal subject comprise one or more of: heart rate, heart rate variability, heart sounds, blood pressure, chest wall displacement, electromyography, electrocardiography, blood oxygenation, respiratory rate, respiratory effort, respiratory cadence, tidal volume, coughing, snoring, sneezing, throat clearing, wheezing, apnea, hypoapnea, physical activity, core body position, peripheral limb position, scratching, vocalizations, rubbing, walking, sleep quality, sleep time, wake time upon sleeping, skin temperature, core body temperature, and a combination thereof.

72. A non-transitory tangible computer-readable medium storing instructions which, when executed by one or more processors, cause the method of claim 57 to be performed.

73. An apparatus for measuring physiological parameters of a mammal subject, comprising:
at least two sensors configured to be in communication with each other wirelessly and bidirectionally in operation, wherein each of the two sensors is bendable such that each of the sensors is configured to have a pre-curved architecture with a non-zero curvature.

74. The apparatus of claim 73, wherein each of the two sensors is at least partially formed by a bendable shape-memory alloy (SMA).
